(19) United States
(12) Patent Application Publication
(10) Pub. No.: US 2012/0240289 A1
(43) Pub. Date: Sep. 20, 2012

(54) METHODS AND MEANS TO ALTER LIPID BIOSYNTHESIS BY TARGETING MULTIPLE ENZYMES TO SUBORGANELLE DOMAINS

(76) Inventors: Ivo Feussner, Gottingen (DE); Mareike Heilmann, Halle (DE)

(21) Appl. No.: 13/503,180

(22) PCT Filed: Oct. 19, 2010
(86) PCT No.: PCT/EP2010/065754

§ 371 Date: Jun. 6, 2012

(30) Foreign Application Priority Data
Oct. 20, 2009 (EP) 09013248.1

(51) Int. Cl. C12N 15/82; A01H 5/00

(52) U.S. Cl. 800/281; 800/298

(57) ABSTRACT

Methods and means are provided to alter lipid biosynthesis in eukaryotic organisms by targeting at least two different polypeptides involved in fatty acid or lipid metabolism towards a similar or the same subdomain of an organelle, such as the endoplasmatic reticulum (ER), through fusion of the polypeptides with a similar or the same heterologous polypeptide targeting the chimeric fusion polypeptide to the mentioned subdomain.

### FIELD OF THE INVENTION

The current invention relates to the field of fatty acid and lipid metabolism. More particularly, methods are provided to alter lipid biosynthesis in eukaryotic organisms by targeting at least two different polypeptides involved in fatty acid or lipid metabolism towards a similar or the same subdomain of an organelle, such as the endoplasmatic reticulum (ER), through fusion of the polypeptides with a similar or the same heterologous polypeptide targeting the chimeric fusion polypeptide to the mentioned subdomain, such as an oleosin. Conveniently, such targeting of the chimeric polypeptides is achieved via recombinant DNA techniques. In a particular embodiment, eukaryotic organism (such as yeasts or plants) are provided which are capable of synthesizing wax esters through expression of a fatty acylCoA reductase and a wax ester synthetase, whereby both polypeptides are operably linked to a similar or the same oleosin polypeptide. By introducing fatty acylCoA reductase and a wax ester synthetase from mouse, with a substrate preference for C12-C18 chain fatty acids, eukaryotic organisms can be provided with a new capability of synthesizing short chain wax esters. Such short chain wax esters have several applications including a potential use as high grade lubrificants.

### BACKGROUND

Lipids and oils, particularly plant oils, represent an important source of nutrients for both human and animal use. Plant oils moreover represent renewable sources of long-chain hydrocarbons that can be used as chemical or fuel feedstocks. The efficient production of oils and lipids, including industrially applicable oils and lipids or oils with specific compositions, in plants is a major goal for plant biotechnology. Although significant advances towards these goals have been achieved, it remains a challenge to provide organisms, such as plants with new enzymes involved in lipid biosynthesis and to obtain efficient expression of the novel catalytic activity and resulting specific lipids. Indeed, the unique types of fatty acids or lipids produced may be incompatible with incorporation into cellular membranes or may interfere with the normal flux of fatty acids into storage oil.

Dyer and Mullen, 2008 (Physiologia Plantarum 132: 11-22) have suggested that the utilization of specific novel enzymes such as diverged FAD enzymes for production in value-added oils in transgenic plants will require placing the enzymes in the correct metabolic context to generate the desired products. These authors further reviewed data concerning the peptide sequences of plant FAD enzymes associated with insertion and maintenance of the enzymes in the ER. This so-called C-terminal retrieval motif could also be identified in enzymes such as DGAT, fatty acid elongases, PDATs and the like.

Shockey et al. 2006 (Plant Cell 18: 2294-2313) described experiments with DGAT proteins demonstrating that these proteins are located in distinct regions or ‘subdomains’ of ER, suggesting that that the C-terminal motifs might be involved in delivery of functionally related proteins to similar regions of ER. Addition of the C-terminal aromatic motifs of DGATs to reporter proteins, however, resulted in localization of these proteins to general ER, rather than subdomains of ER, indicating that other portions of the proteins are required for their localization and/or organization into functionally distinct ER subdomains.

The prior art therefore remains defective in providing a solution to engineer functionally related novel enzymes in such a way as to target them to specific subdomains of the ER and efficiently synthesize the novel product through concerted action of the novel enzymes.

One embodiment of the current invention relates to the production of short chain wax esters in plants. Wax esters, the main constituents of waxes, are known to be neutral lipids, consisting of long-chain fatty acids, which are esterified with long chain fatty alcohols. The two molecular species included in a wax ester can differ in chain length and number of double bonds, which leads to a broad variety of different esters. Wax esters are highly hydrophobic and polar due to the lack of a head group and, for this reason, insoluble in water. Further, they usually are ductile at room temperature and rather viscous after melting. Additional, long chain wax esters are known to have a very low volatility (Vrkoslav and Mikova, 2009). Wax esters are widespread in nature and can be found in plants, microorganisms and animals, mainly coating their surface to prevent water loss, abrasion and infection (Cheng and Russell, 2004a).

The Jojoba plant (Simmondsia chinensis) is unique based on its ability to use liquid wax esters as energy storage instead of triacylglycerols (TAG) in its seeds. It is therefore of high interest in terms of renewable sources of high quality oil. The jojoba wax esters consist of 18:1, 20:1 and 22:1 fatty acids linked to 20:1, 22:1 and 24:1 fatty alcohols, meaning that the wax contains C38 to C44 esters with one double bond in each alkyl moiety, respectively. However, jojoba plants grow only in restricted geographic areas and the seed yield of these plants is not sufficient to allow the use of jojoba was esters in high quantities.

Wax esters fulfill a variety of diverse and important biological functions. They can act as protection against desiccation, UV light and pathogens by coating plant leaf surfaces (cuticula) with a thin layer, respectively. Waxes can also function as water—proof layer to the feathers of birds. Furthermore, wax esters may have structural functions for instance while being secreted by bees to construct their honeycombs. As described above, wax esters also function as energy storage in the jojoba plant and other members of the family Euphorbiaceae. These plants contain mainly wax esters in their seeds instead of the usual triacylglycerol (TAG). Sperm whales produce spermaceti oil in their head cavities, which is needed for regulation of buoyancy, sound transmission and echo location. Mammals produce wax esters as well, mostly in the sebaceous glands, which secrete the esters together with further substances (sebum) onto the surface of the skin and eyes.

Wax esters have a multitude of important technical applications in a variety of areas, including medicine, cosmetics, and food industries, as well as a more traditional use as lubricants. A traditional source for wax esters was the spermaceti oil produced by spermaceti whales. These esters have been used extensively in lubrication and transmission fluids.

One goal of the current invention is to provide a solution to the production in sufficient amounts from a readily renewable source, of types of wax esters with high value due to lower melting points and enhanced lubrication properties impaired by shorter chain length and/or hydroxyl and methyl substitutions. Short-chain wax esters are of particular interest, because they can be used at extremely low temperatures and high pressures without any disaggregation.

### BRIEF DESCRIPTION OF THE DRAWINGS

FIG. 1: Alignment of FAR enzymes of various organisms.

Alignment of the two fatty acid reductases (FAR) of Mus musculus MmFAR1 (protein ID Mm.206919) and MmFAR2 (protein ID Mm.475174) with diverse FARs from Arabidopsis thaliana AtFAR (At3g11980); AtFAR1 (At5g22500); AtFAR5 (At3g44550); AtFAR6 (Atg56700) and S. chinensis (Jojoba), SchFAR (Accession No. AF149918).

FIG. 2: Localization of various mCherry fusion proteins in onion epidermial cells.

Onion epidermal peels were bombarded with DNA encoding different mCherry fusion proteins. The bombarded cells were incubated over night at room temperature and then analyzed via fluorescence microscopy.
A-C, Localization analysis of mCherry-FAR1 with the peroxisomal marker MDH: A, Onion epidermal cell expressing mCherry-MmFAR1 B, onion epidermal cells coexpressing the peroxisomal marker MDH with mCherry-MmFAR1 and C, overlay of A and B.
D-F, Localization analysis of mCherry-FAR1c with the peroxisomal marker MDH: D show onion epidermal cells expressing mCherry-MmFAR1c. E, Co-expression of the peroxisomal marker MDH. I, Overlay of mCherry-MmFAR lc fluorescence image with the MDH fluorescence image.

FIG. 3: TLC analysis of fatty alcohol products.

Total lipid extracts from yeast expressing either the empty vector (control), FAR1, mCherry-MmFAR1 or the C-terminal truncated version, mCherry-FAR1c. Standards: DAG, diacylglycerol; 16:0-OH as standard for fatty alcohols; FFA, free fatty acids; WE, wax ester; ST, sterols. Developing solvent: hexane:diethyl ether:acetic acid (65:35:1 v/v/v). This experiment was performed three times, once also using a different yeast strain (INVSc1).

FIG. 4: Oleo3-mCherry-MmFAR1 and Oleo3-mCherry-MmFAR1c constructs in onion epidermal cells.

Onion epidermal peels were bombarded with DNA encoding different mCherry fusion proteins. The bombarded cells were incubated over night at room temperature and then analyzed via fluorescence microscopy. A-C: Localization analysis of Oleo3-mCherry-FAR1c. D: Onion epidermal cells expressing either Oleo3-mCherry-MmFAR1c or, E co-expressing either the peroxisomal marker MDH with Oleo3-mCherry-MmFAR1c. F overlay of D and E and G and H, respectively. Bombardment was performed twice and in each experiment about 50 cells were observed. E: Enlarged detail of the overlay of Oleo3-mCherry-MmFAR1c co-expressed with MDH. The YFP fluorescent spots represent the peroxisomes visualized by the YFP tagged MDH. The red spots represent the position of the FAR1c constructs visualized by mCherr

FIG. 5: TLC performed with mCherry-MmFAR1+MmWS, mCherry-MmFAR1c+MmWS, Oleo3mCherry-MmFAR1c+MmWS and Oleo3mCherry-MmFAR1c+Oleo-MmWS expressed for 48 h

A: Sterols, a TAG-mixture, 16:0 fatty acid, 16:0-OH fatty alcohol and wax esters (WE) were used as standards. A control containing only the empty vector, the vector containing mCherry-FAR1+WS, the vector containing mCherry-Far1c+WS, the vector containing the Oleo3-mCherry-FAR1c+WS and the vector containing the Oleo3-mCherry-MmFAR1c+Oleo3-WS constructs were tested. Developing solvent: hexane:diethyl ether:formic acid (90:10:1 v/v/v).

FIG. 6: Diagram shows the quantification of wax ester accumulation in the samples 1-4 of FIG. 5.

FIG. 7: Overview of the MmFAR2, MmFAR1 and MmWS constructs.

A, original unmodified MmFAR1 enzyme tagged to mCherry fused to A. thaliana oleosin 3.

B, truncated version of MmFAR1, MmFAR1c, tagged to mCherry fused to A. thaliana oleosin 3. MmFAR1c lacks the C-terminal putative transmembrane domains.

C MmWS fused to A. thaliana oleosin 3.

FIG. 8: Nile red stained onion epidermis cells.

Onion epidermal peels, bombarded with DNA encoding YFP-Oleosin3, or onion epidermal peels not expressing any transgene were stained with Nile red. A: Localization of Oleo3-YFP. B: Localization of Nile Red staining. C: Overlay of A and B. D: Localization of Nile Red staining in untransformed onion cells. E: Magnification of C.

FIG. 9: Localization of Oleo3-mCherry-MmFAR1c in onion epidermal cells co-expressed with pMDH or YFP-Oleo3.

Onion epidermal peels were co-bombarded with DNA encoding the Oleo3-mCherry-MmFAR1c fusion protein together with DNA encoding the peroxisomal marker MDH-YFP or with the oil body marker Oleo3-YFP. A and E: Localization of Oleo3-mCherry-MmFAR1c. B: Localization of MDH-YFP (peroxisome). C: Overlay of A and B. D: Magnification of C. F: Localization of Oleo3-YFP (oil body). G: Overlay of E and F. H: Magnification of G.

FIG. 10: Co-localization of Oleo3-mCherry-FAR1c with Oleo3-YFP-WS in onion epidermis cells.

Onion epidermal peels were co-bombarded with DNA encoding the Oleo3-mCherry-MmFAR1c fusion protein together with DNA encoding the Oleo3-YFP-WS fusion protein. A: Localization of Oleo3-mCherry-MmFAR1c. B: Localization of Oleo3-YFP-WS. C: Overlay of A and B. D: Magnification of C.

FIG. 11: Quantification of WE accumulation by GC-MS after co-expression of unmodified and modified MmFAR1 and MmWS.

mCherry-MmFAR1+YFP-MmWS, mCherry-MmFAR1c+YFP-MmWS, Oleo3mCherry-MmFAR1c+MmWS and Oleo3mCherry-MmFAR1c+Oleo3-YPF-MmWS were expressed in yeast for 72 h at 30° C. WE accumulation was quantified by GC-MS. Experiment represents data from 6 independent clones in each case.

FIG. 12: Individual expression of different MmFAR1-versions in yeast.

mCherry-MmFAR1, mCherry-MmFAR1c and Oleo3-mCherry-MmFAR1c were expressed in yeast for 72 h at 30° C. Three independent clones were tested. The fatty acids were analyzed with TLC as described for FIG. 5.

### SUMMARY OF THE INVENTION

In one embodiment of the invention, a method is provided for modulating lipid biosynthesis in a eukaryotic organism comprising the step of providing to cells of said organism a multitude of chimeric proteins wherein each of said chimeric proteins comprises
a. a heterologous polypeptide targeting said proteins to a similar subdomain of an organelle in said cell operably linked to
b. a polypeptide involved in fatty acid metabolism or lipid metabolism.

The heterologous polypeptide may comprises a polypeptide sequence of an oilbody protein or a targeting part thereof, or the heterologous polypeptide comprises a polypeptide sequence selected from an oleosin, such as a polypeptide sequence having at least 80% homology to the amino acid sequence of SEQ ID No SEQ ID No 10 from amino acid 1 to amino acid 143, or an endoplasmatic retrieval signal from fatty acid desaturases, the N-terminal domain of LBLOX or the N-terminal domain of PLA. The polypeptide involved in fatty acid metabolism or lipid metabolism may be selected from an acyl-CoA synthetase, a glycerol-phosphate acyltransferase, an O-acyltransferase, a lyso-phosphatidic acid acyltransferase, a phosphatidic acid phosphatase, a diacylglycerol acyltransferase, an oleate desaturases, a linoleate desaturases, an acyl-CoA hydroxylase, an acyl-lipid hydroxylase, a fatty acid epoxidase, a phospholipid:sterol acyltransferase, a phospholipid:diacylglycerol acyltransferase, a diacylglycerol transacylase, a lysophosphatidylcholine acyltransferase, a phosphatidylcholine:diacylglycerol cholinephosphotransferase, an acyl-CoA elongase, an acyl-lipid elongase, a phosphatidylglycerol-phosphate synthetase, a phosphatidylglycerol-phosphate phosphatase, a CDP-diacylglycerol synthetase, a phosphatidylinositol synthase, a phosphatidylserine synthase, a choline kinase, an ethanolamine kinase, a CDP-choline synthetase, a CDP-ethanolamine synthetase, a phosphatidylserine decarboxylase, a lipoxygenase, a phospholipase, a lipase, a carboxylesterase, a fatty alcohol reductase, a wax ester synthase, a bifunctional acyltranferases/wax synthase, a ketoacyl-CoA synthase, a ketoacyl-CoA reductase, a hydroxylacyl-CoA dehydrase, an enoyl-CoA reductase, an alcohol-forming fatty acyl-CoA reductase, an aldehyde-forming fatty acyl-CoA reductase, an aldehyde decarbonylase, a wax ester hydrolase, a glycerol-3-P-dehydrogenase, a CDP-choline:1,2-diacylglycerol cholinephosphotransferase, an oxidase, a ketosphinganine reductase, a ceramide synthase, an acylglycerophosphorylcholine acyltransferase, an acylglycerol-phosphate acyltransferase, a phosphoethanolamine N-methyltransferase, a ceramide sphingobase desaturase, a glucosylceramide synthase, a acyl-ceramide synthase, a triacylglycerol lipase, a monoacylglycerol lipase, an acyl-CoA oxidase, an hydroxyacyl-CoA dehydrogenase, a dienoyl-CoA reductase, a fatty acid omega-alcohol oxidase, a monoacylglycerol lipase, an acyl-CoA oxidase, a hydroxyacyl-CoA dehydrogenase, a dienoyl-CoA reductase, a fatty acid omega-alcohol oxidase, a fatty acid/acyl-CoA transporter, a acyl-CoA dehydrogenase, a diacylglycerol-phosphate kinase, a lysophosphatidic acic phosphatase, a peroxygenase; a Δ4-desaturase; a Δ5-desaturase, a Δ6-desaturase; a Δ9-desaturase, a a Δ12-desaturase or a Δ15-desaturase.

In a particular embodiment of the invention one chimeric protein may comprise a fatty acyl-CoA reductase and another chimeric protein may comprise a wax ester synthase such as a fatty acyl-CoA reductase with a substrate preference for fatty acyls within the range C16 to C18; and a wax ester synthase has a substrate preference for fatty alcohols and fatty acyl-CoA within the range C16-C18. Such fatty acyl-CoA reductase may derived from mouse and preferably has an amino acid sequence having about 80% sequence identity to the amino acid sequence of SEQ ID 6. Such wax ester synthase may be derived from mouse and preferably has an amino acid sequence having about 80% sequence identity to the amino acid sequence of SEQ ID 12.

Conveniently, the chimeric proteins may be expressed from one or more DNA constructs comprising the following operably linked DNA fragments:
a. A promoter functional in cells of said eukaryotic organism
b. A DNA region encoding said chimeric protein
c. A transcription termination and/or polyadenylation region.

The eukaryotic organism may be selected from a plant, such as rapeseed (Brassica spp.), flax (Linum usitatissimum), safflower (Carthamus tinctorius), sunflower (Helianthus annuus), maize or corn (Zea mays), soybean (Glycine max), mustard (Brassica spp. and Sinapis alba), crambe (Crambe abyssinica), eruca (Eruca saiva), oil palm (Elaeis guineeis), cottonseed (Gossypium spp.), groundnut (Arachis hypogaea), coconut (Cocus nucifera), castor bean (Ricinus communis), coriander (Coriandrum sativum), squash (Cucurbita maxima), Brazil nut (Bertholletia excelsa) or jojoba (Simmondsia chinensis), or an animal, a fungus or a yeast.

In another embodiment of the invention, a method is provided for producing selected wax esters in plants comprising the steps of providing to cell of said plant organism at least two chimeric proteins wherein the first of said chimeric proteins comprises a first heterologous polypeptide targeting said protein to a subdomain of an organelle in said cell operably linked to a fatty-acyl CoA reductase and wherein the second of said chimeric proteins comprises a second heterologous polypeptide targeting said protein to said subdomain of said organelle in said cell operably linked to a wax ester synthase.

It is another object of the invention to provide a composition of lipids derived from a plant obtained according to the methods herein described.

Yet another object of the invention is to provide a non-human eukaryotic organism comprising at least a first and a second DNA construct,
a. said first DNA construct comprising:
   i. a first promoter functional in cells of said eukaryotic organism
   ii. a first DNA region encoding a first chimeric protein comprising
      1. A DNA region encoding a first heterologous polypeptide targeting said proteins to a particular subdomain of an organelle in said cell; operably linked to
      2. A DNA region encoding a first polypeptide involved in fatty acid metabolism or lipid metabolism;
   iii. a first transcription termination and/or polyadenylation region; and
b. said second DNA construct comprising
   iv. a second promoter functional in cells of said eukaryotic organism
   v. a second DNA region encoding a second chimeric protein comprising
      1. A DNA region encoding a second heterologous polypeptide targeting said proteins to said particular subdomain of an organelle in said cell; operably linked to
      2. A DNA region encoding a second polypeptide involved in fatty acid metabolism or lipid metabolism;
   vi. a first transcription termination and/or polyadenylation region;
c. wherein said first and second heterologous polypeptide may be the same or different and wherein said first and second polypeptide involved in fatty acid metabolism or lipid metabolism are different polypeptides.

The invention further provides a method to alter the subcellular location of an animal fatty-acyl CoA reductase comprising the steps of deleting the C-terminal amino acid sequences having the amino acid sequence of SEQ ID 2 from amino acid 467 to amino acid 515 or an amino acid sequence having at least 80% sequence identity thereto.

### DESCRIPTION OF DIFFERENT EMBODIMENTS OF THE INVENTION

The current invention is based on the inventors observation that, when expressing in a eukaryotic organism, a first enzyme involved in lipid biosynthesis such as a fatty acyl CoA reductase and a second enzyme involved in lipid biosynthesis such as a wax ester synthase, which are supposed to act in a concerted manner to produce the desired wax esters, the amount of desired end product is much higher when both enzymes are fused to a polypeptide targeting the fused enzymes to a similar subdomain of the endoplasmatic reticulum (ER), such as the oleosin 3 polypeptide, than when the enzymes are not targeted to the same subdomain in the ER.

Accordingly, in a first embodiment, the invention provides a method for modulating lipid biosynthesis in a eukaryotic organism comprising the step of providing to cells of the eukaryotic organism at least one chimeric protein, but preferably a multitude of chimeric proteins wherein each of said chimeric proteins comprises a polypeptide involved in fatty acid metabolism or lipid metabolism operably linked to a heterologous polypeptide targeting the chimeric proteins to the same subdomain of an organelle, such as the ER.

As used herein “a polypeptide targeting the chimeric proteins to a subdomain of an organelle” refers to a polypeptide having a specific amino acid sequence which results in the location of the associated polypeptide involved in lipid or fatty acid biosynthesis to a subdomain of an organelle of the eukaryotic cell, such as a subdomain of the ER where (the relevant part of) lipid or fatty acid biosynthesis is occurring. In doing so, the associated polypeptides involved in lipid or fatty acid biosynthesis are brought in the correct metabolic context, so that the desired end-product is produced in an efficient manner.

A “heterologous” polypeptide targeting the chimeric proteins to a subdomain of an organelle refers to the fact that the targeting polypeptide is not occurring naturally within the context of the polypeptide involved in lipid or fatty acid biosynthesis with which it is associated in the context of the current invention. In other words, a novel combination, not normally occurring in nature, is made between the targeting polypeptide and the polypeptide involved in lipid or fatty acid biosynthesis.

In one embodiment of the invention, the heterologous targeting polypeptide may comprise the amino acid sequence of an oleosin.

Oleosin are proteins associated with oil bodies, wherein triacylglycerides (TAG) are sequestered. Oil bodies (OB) are spherical organelles 0.2-2.0 mm in diameter with a simple structure consisting of a TAG core encased in a half-unit membrane consisting of phospholipids and a few different proteins of which oleosin form the majority. The sequence/structure of oleosins consists of three distinct domains: first, an amino-terminal domain, which may be either hydrophilic or hydrophobic; second, a central 70-77 amino acid hydrophobic domain with a conserved proline knot in the center of the domain; third, a carboxyterminal amphipathic domain of variable length. The signature characteristic of the oleosins is that they all possess the central hydrophobic domain. The central hydrophobic region constitutes the longest continuous region of hydrophobic amino acids known in any protein and is unique to these proteins. This hydrophobic domain has been modeled as an anti-parallel helical structure anchored in the TAG core with the proline knot reversing the direction of the polypeptide. The C-terminal amphipathic domain is largely conserved as an amphipathic alpha-helical structure but the sequence of this domain is highly variable. Oleosin proteins from one species will correctly associate with forming OBs of another species; for example, the soybean oleosin gene transferred into Brassica napus resulted in soybean oleosin being correctly expressed in seed development and the soybean oleosin protein was inserted into the Brassica OBs as a mixed population of proteins with the intrinsic Brassica oleosin.

Oleosins suitable for the purpose of the invention are known in the art and the following is a list of amino acid sequence and nucleotide sequences encoding such amino acid sequences: A84654 Arabidopsis thaliana probable oleosin; AAA87295 Arabidopsis thaliana oleosin (Gene L40954); AAC42242 Arabidopsis thaliana oleosin (Gene AC005395);AAF015421 Arabidopsis thaliana putative oleosin (Gene AC009325); AAF 697121 Arabidopsis thaliana F27J15.22 (Gene AC016041); AAK96731 Arabidopsis thaliana oleosin-like protein (Gene AY054540); AAL143 85 Arabidopsis thaliana AT5g 40420/MPO12_{—}130 oleosin isoform (Gene AY057590); AAL24418 Arabidopsis thaliana putative oleosin (Gene AY059936); AAL473566 Arabidopsis thaliana oleosin-like protein (Gene AY064657); AAM10217 Arabidopsis thaliana putative oleosin (Gene AY081655); AAM47319 Arabidopsis thaliana AT5g40420/MPO12_{—}130 olesoin isoform (Gene AY113011); AAM63098 Arabidopsis thaliana oleosin isoform (Gene AY085886); AA022633 Arabidopsis thaliana putative oleosin (Gene BT002813); AA022794 Arabidopsis thaliana putative oleosin protein (Gene BT002985); AA042120 Arabidopsis thaliana putative oleosin (Gene BT004094); AA050491 Arabidopsis thaliana putative oleosin (Gene BT004958); AA063989 Arabidopsis thaliana putative oleosin (Gene BT005569); AAQ22658 Arabidopsis thaliana At4g25140 (Gene BT010189.1); AAQ56108 Arabidopsis lyrata susp. Lyrata Oleosin (Gene AY292860); BAA97384 Arabidopsis thaliana oleosin-like (Gene AB023044); BAB02690 Arabidopsis thaliana oleosin-like protein (Gene AB018114); BAB11599 Arabidopsis thaliana oleosin isoform 21K (Gene AB006702); BAC42839 Arabidopsis thaliana putative oleosin protein (Gene AK118217); BAD94320 Arabidopsis thaliana oleosin (Gene AK220898.1); CAA44225 Arabidopsis thaliana oleosin (Gene X62353); CAA63011 Arabidopsis thaliana oleosin type 4 (Gene X91918); CAA63022 Arabidopsis thaliana oleosin type 2 (Gene X91956); CAA90877 Arabidopsis thaliana oleosin (Gene Z54164); CAA90878) Arabidopsis thaliana oleosin (Gene Z54165); CAB36756 Arabidopsis thaliana oleosin 18.5 K (Gene AL035523); CAB79243 Arabidopsis thaliana oleosin, 18.5 K (Gene AL161562); CAB87945 Arabidopsis thaliana oleosin-like Protein (Gene ALI63912); P29525 Arabidopsis thaliana oleosin 18.5 kDa (Gene X62353, CAA44225,AL0355 23, CAB36756, CAB79423, Z17738, S22538); Q39165 Arabidopsis thaliana Oleosin 21.2 kDa (Oleosin type 2) (Gene L40954, AAA87295, X91956, CAA63022, Z17657, AB006702, BAB11599,AY057590, AAL14385, S71253); Q42431 Arabidopsis thaliana oleosin 20.3 kDa (Oleosin type 4) (Gene Z54164, CAA90877, X91918, CAA63011, AB018114, BAB02690, AY054540, AAK96731, AY064657, A.AL47366, AY085886, AAM 63098, Z27260, Z29859, S71286); Q43284 Arabidopsis thaliana Oleosin 14.9 kDa. (Gene Z54165, CAA90878, AB023044, BAA97384, Z27008, CAA81561);S22538 Arabidopsis thaliana oleosin 18.5K; 5751253 oleosin, 21K; 571286 Arabidopsis thaliana 20K; T49895 Arabidopsis thaliana oleosin like protein; AAB22218 Brassica napus oleosin napII; AAB22219 Brassica napus oleosin napI; AAD24547 Brassica napus oleosin; AAK38471 Brassica napus oleracea putative oleosin (Gene AY028608.1); AAK38472 Brassica oleracea putative oleosin (Gene AY028608.1) ; AAK38473 Brassica oleracea putative oleosin (Gene AY028608.1); AAK38474 Brassica oleracea putative oleosin (Gene AY028608.1); AAK38475 Brassica oleracea putative oleosin (Gene AY028608.1); AAW70038 Brassica rapa oleosin-like protein (Gene AY747625.1); CAA41064 Brassica napus oleosin Nap-II(Gene X58000.1); CAA43941 Brassica napus oleosin BN-III (Gene X63779); CAA45313 Brassica napus oleosin BN-V (Gene X63779); CAA57544 Brassica napus oleosin (Gene X82019.1); CAA57545 Brassica napus oleosin (X82020.1); CAA64800 Brassica napus oleosin-like protein (Gene X95554.1); CAA64801 Brassica napus oleosin -like protein (Gene X95555.1); CAA64802 Brassica napus oleosin-like protein (Gene X95556.1); CAA64803 Brassica napus oleosin-like protein (Gene X95557.1); CAA64804 Brassica napus oleosin-like protein (Gene X95558.1); CAA64805 Brassica napus oleosin-like protein (Gene X95559.1); CAA64806 Brassica napus oleosin-like protein (Gene X95560.1); CAA70173 Brassica napus oleosin-like protein (Gene Y08986.1); P29109 Brassica napus oleosin Bn_V (Gene X63779, CAA45313, S25089); P29110 Brassica napus oleosin Bn-III (Gene X61937, CAA43941, S22475); P29111 Brassica napus oleosin NAP-II (Gene X58000, CAA41064, S70915); P29526 Brassica napus oleosin C98 (Gene X67142.1, CAA47623.1; S24960); S13494 Brassica napus oleosin NAP-I; S22475 Brassica napus oleosin BN-III; S25089 Brassica napus oleosin BN-IV; S50195 Brassica napus oleosin; S70915 Brassica napus oleosin Nap-II; T08134 Brassica napus oleosin; 1803528A Brassica napus oleosin; 2009397 Brassica napus oleosin; AAB01098 Daucus carota oleosin; T14307 carrot oleosin; A35040 Zea mays oleosin 18; AAA67699 Zea mays oleosin KD18 (Gene J05212); AAA68065 Zea mays oleosin 16 Kda (Gene U13701); AAA68066 Zea mays oleosin 16 Kda (Gene U13702); P13436 Zea mays oleosin ZM-1 (oleosin 16kD) (Gene U 3701, AAA68065, M17225, AAA33481, A29788); P21641 Zea mays oleosin Zm-II (oleosin 18 KDa)(Gene J05212, AAA67699, A35040); S52029 Zea mays oleosin 16; 552030 Zea mays oleosin 17. All these nucleotide and amino acid sequences are herein incorporated by reference.

In another embodiment of the invention, the heterologous targeting polypeptide may comprise the amino acid sequence of the N-terminal domain of LBLOX (lipid body lipoxygenase) or the N-terminal domain of PLA (phospholipase A2) as described in WO01/29227.

In yet another embodiment of the invention, the heterologous targeting polypeptide may comprise the amino acid sequence of the ER retrieval sequences as described in Dyer and Mullen, 2008 (Physiologia Plantarum 132: 11-22). One such ER retrieval sequence C-terminal dilysine ER retrieval motif as can be found in FAD3 polypeptides. Another ER retrieval sequence comprises a C-terminal aromatic-rich motif similar to a 1—WxxxW—motif. Yet another ER retrieval sequence comprises the consensus sequence consisting of Φ-x-x-K/R/D/E-ΦCOOH, where Φ is any large, hydrophobic amino acid and —x— is any amino acid.

The heterologous targeting polypeptide may also comprise an ER retention signal (K/HDEL*), or an ER-retrieval signal for membrane bound proteins (KKxx*) or —KK—COOH or —KKXX—COOH or —KXKXX—COOH motif. The heterologous targeting polypeptide may also comprise to the transmembrane segment of α-2,6-sialyltransferase (particularly the first 44 or 52 amino acids thereof; Munro et al. 1991, EMBO Journal, 10: 3577-3588); the signal anchor sequence from human galactosyl transferase (particularly the first 60 amino acids thereof) or the signal anchor sequence from the Arabidopsis homologue of the yeast HDEL receptor (AtERD2) (Saint-Jore et al., 2002, The Plant Journal, 29: 661-678), the signal anchor sequence from β1,2-xylosyltransferase protein (particularly the first 36 amino acids thereof; Pagny et al., 2003, The Plant Journal 33: 189-203) or the signal anchor sequences of N-acetyl-glucosaminyl transferase I (particularly the first 77 amino acids thereof; Essl et al. 1999, FEBS Lett. 453:169-173).

In one embodiment of the invention, the heterologous targeting polypeptide may be a polypeptide having at least 80% sequence identity to oleosin 3 of Arabidopsis thaliana (SEQ ID No 10 from amino acid 1 to amino acid 143). It will be clear that a polypeptide having at sequence identity of more than at least 80%, such as 85%, 90%, 95% or 100% can also be used to the same effect.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as GAP which is part of the Wisconsin Package Version 10.1 (Genetics Computer Group, Madision, Wis., USA) using the default scoring matrix with a gap creation penalty of 50 and a gap extension penalty of 3.

It will be clear to the skilled artisan that the heterologous targeting peptide present in the different chimeric proteins may be either the same or may be different, provided that it targets the chimeric proteins to the same suborganelle domain in the eukaryotic cell.

It will also be clear to the skilled artisan that it may be sufficient to provide only one chimeric gene comprising a heterologous targeting polypeptide operably linked to a polypeptide involved in fatty acid or lipid biosynthesis, if the other components of the multitude of proteins involved in lipid or fatty acid metabolism provided to the eukayotic cells already comprise an endogenous targeting polypeptide targeting that other protein to the same subdomain of the organelle.

As used herein “a polypeptide involved in fatty acid metabolism or lipid metabolism” refers to a polypeptide, preferably a polypeptide with catalytic activity, which is actively contributes to the fatty acid metabolism or lipid metabolism (i.e. biosynthesis or degradation) in an organism. Such polypeptides include but are not limited to an acyl-CoA synthetase, a glycerol-phosphate acyltransferase, an O-acyltransferase, a lyso-phosphatidic acid acyltransferase, a phosphatidic acid phosphatase, a diacylglycerol acyltransferase, an oleate desaturases, a linoleate desaturases, an acyl-CoA hydroxylase, an acyl-lipid hydroxylase, a fatty acid epoxidase, a phospholipid:sterol acyltransferase, a phospholipid:diacylglycerol acyltransferase, a diacylglycerol transacylase, a lysophosphatidylcholine acyltransferase, a phosphatidylcholine:diacylglycerol cholinephosphotransferase, an acyl-CoA elongase, an acyl-lipid elongase, a phosphatidylglycerol-phosphate synthetase, a phosphatidylglycerol-phosphate phosphatase, a CDP-diacylglycerol synthetase, a phosphatidylinositol synthase, a phosphatidylserine synthase, a choline kinase, an ethanolamine kinase, a CDP-choline synthetase, a CDP-ethanolamine synthetase, a phosphatidylserine decarboxylase, a lipoxygenase, a phospholipase, a lipase, a carboxylesterase, a fatty alcohol reductase, a wax ester synthase, a bifunctional acyltranferases/wax synthase, a ketoacyl-CoA synthase, a ketoacyl-CoA reductase, a hydroxylacyl-CoA dehydrase, an enoyl-CoA reductase, an alcohol-forming fatty acyl-CoA reductase, an aldehyde-forming fatty acyl-CoA reductase, an aldehyde decarbonylase, a wax ester hydrolase, a glycerol-3-P-dehydrogenase, a CDP-choline:1,2-diacylglycerol cholinephosphotransferase, an oxidase, a ketosphinganine reductase, a ceramide synthase, an acylglycerophosphorylcholine acyltransferase, an acylglycerol-phosphate acyltransferase, a phosphoethanolamine N-methyltransferase, a ceramide sphingobase desaturase, a glucosylceramide synthase, a acyl-ceramide synthase, a triacylglycerol lipase, a monoacylglycerol lipase, an acyl-CoA oxidase, an hydroxyacyl-CoA dehydrogenase, a dienoyl-CoA reductase, a fatty acid omega-alcohol oxidase, a monoacylglycerol lipase, an acyl-CoA oxidase, a hydroxyacyl-CoA dehydrogenase, a dienoyl-CoA reductase, a fatty acid omega-alcohol oxidase, a fatty acid/acyl-CoA transporter, a acyl-CoA dehydrogenase, a diacylglycerol-phosphate kinase, a lysophosphatidic acic phosphatase, a peroxygenase; a Δ4-desaturase; a Δ5-desaturase, a Δ6-desaturase; a Δ9-desaturase, a Δ12-desaturase or a Δ15-desaturase.

These polypeptides are well known in the art and may be derived from animal sources, plant sources, bacterial sources, algal sources or fungal sources. Enzymes relevant for the production of polyunsaturated fatty acids may be the ones described in WO2006/064317, WO2008/104559, WO2008/076987, WO2007/136877 or WO2007/106728 (herein incorporated by reference). Enzymes relevant for modulating oil content include Arabidopsis DGAT1 described in patent application PCT/CA99/01202, Tropaeolum DGAT1 described in patent application pct/ca2007/001225, Umbelopsis ramanniana DGAT2; diacylglycerol acyltransferase 2 genes and proteins encoded thereby from algae described WO2009/085169; pyruvate kinase genes described in WO2008/135467, plant sucrose synthase like proteins described in WO2006/133166 or yeast glycerol-3-phosphate dehydrogenase genes described in WO2003/095655.

In one embodiment of the invention, one of the polypeptides involved in fatty acid or lipid metabolism comprise a fatty acyl-CoA reductase and another comprises a wax ester synthase. As used herein, a fatty acyl-Co reductase is an enzyme reducing a fatty acyl CoA to the corresponding fatty alcohol using electrons from NADPH cofactor, and thereby cleaving the thioester bond in the fatty acyl-CoA. Fatty acyl-CoA reductases having a substrate preference for C12-C20 fatty acyl-CoA molecules are especially suited for the methods according to the invention. As an example, the current invention employs fatty acyl-CoA reductase from mouse. Accordingly, in one embodiment of the invention, one of the chimeric proteins comprises a polypeptide involved in fatty acid or lipid biosynthesis comprising an amino acid sequence having at least 80% sequence identity to the amino acid sequence of SEQ ID No 2. During the experiment leading to the current invention, the inventors have observed that the mouse fatty acyl-CoA reductase comprises a C-terminal domain (amino acids 467 to 515 of SEQ ID No 2) which is involved in the location of the mouse fatty acyl-CoA reductase to the peroxisomes and which can be deleted (and preferably should be deleted when retargeting the fatty acyl-CoA reductase in accordance with the invention) without interfering with the catalytic activity of the fatty acyl-CoA reductase. Thus, in another embodiment of the invention one of the chimeric proteins comprises a polypeptide involved in fatty acid or lipid biosynthesis comprising an amino acid sequence having at least 80% sequence identity to the amino acid sequence of SEQ ID No 6. Again, it will be clear that a polypeptide having at sequence identity of more than at least 80%, such as 85%, 90%, 95% or 100% can also be used to the same effect.

As used herein, a wax ester synthase is an enzyme catalyzing the trans-esterification of a fatty alcohol and a fatty acyl-CoA producing a wax monoester. CoASH is released and the energy linked to the thioester bond is used to create the new ester linkage.

In higher plants, mammals and bacteria, ester biosynthesis is catalyzed by one of three classes of wax synthase (WS) enzymes: jojoba-type WS, mammalian WS, and WS/diacylglycerol O-acyltransferases (DGAT) bifunctional enzymes (Jetter and Kunst, 2008). Jojoba-type WS uses a wide range of saturated and unsaturated acyl CoAs ranging from C14 to C24, with 20:1 as the preferred acyl and 18:1 as the preferred alcohol substrate (Lardizabal et al., 2000). Mammalian WS enzymes do not have homologues in plants, and have highest activities with C12-C16 acyl-CoAs and alcohols shorter than C20 (Cheng and Russell, 2004b). It is generally accepted that WS are localized to the ER membrane. WS catalyzes the transesterification of a fatty alcohol and a fatty acyl-CoA. The mouse WS in particular is found in the preputial glands and the eyelids and prefers mainly C16:0, C18:1, and C18:2 fatty alcohols and fatty acyl-CoAs in the following order: C16:1 >C18:1 >C16:0 >C20:0 >C14:0. This shows, that wax monoesters emerging in mouse cells are notably short and therefore of great industrial interest (see 1.3; Cheng and Russell, 2004b; Lassner et al.,1999). In one embodiment of the invention, the wax ester synthase may comprise an amino acid sequence having at least 80% sequence identity to the amino acid sequence of SEQ ID No 12.

The chimeric proteins according to the invention are conveniently provided to the eukaryotic cells by expressing them from one or more DNA constructs comprising a promoter functional in cells of said eukaryotic organism operably linked to a DNA region encoding the chimeric protein and a transcription termination and/or polyadenylation region. It will be clear to the skilled artisan that the DNA constructs may be organized as an operon whereby the expressed (i.e. the transcribed and translated region) may be under control of a single promoter, or as separate transcription units, whereby each coding DNA region is under control of a single promoter.

In one embodiment of the invention, the promoter is a plant expressible promoter. As used herein, the term “plant-expressible promoter” means a DNA sequence which is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Harpster et al., 1988 Mol. Gen. Genet. 212, 182-190), the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al., 1996, The Plant Cell 8, 15-30), stem-specific promoters (Keller et al., 1988, EMBO J. 7, 3625-3633), leaf specific promoters (Hudspeth et al., 1989, Plant Mol Biol 12, 579-589), mesophyl-specific promoters (such as the light-inducible Rubisco promoters), root-specific promoters (Keller et al.,1989, Genes Devel. 3, 1639-1646), tuber-specific promoters (Keil et al., 1989, EMBO J. 8, 1323-1330), vascular tissue specific promoters (Peleman et al., 1989, Gene 84, 359-369), stamen-selective promoters (WO89/10396, WO 92/13956), dehiscence zone specific promoters (WO 97/13865) and the like.

Seed specific promoters are well known in the art, including the USP promoter from Vicia faba described in DE10211617; the promoter sequences described in WO2009/073738; promoters from Brassica napus for seed specific gene expression as described in WO2009/077478; the plant seed specific promoters described in US2007/0022502; the plant seed specific promoters described in WO03/014347; the seed specific promoter described in WO2009/125826; the promoters of the omega_{—}3 fatty acid desaturase family described in WO2006/005807 and the like.

Methods to obtain transgenic plants are not deemed critical for the current invention and any transformation method and regeneration suitable for a particular plant species can be used. Such methods are well known in the art and include Agrobacterium-mediated transformation, particle gun delivery, microinjection, electroporation of intact cells, polyethyleneglycol-mediated protoplast transformation, electroporation of protoplasts, liposome-mediated transformation, silicon-whiskers mediated transformation etc. The transformed cells obtained in this way may then be regenerated into mature fertile plants.

The obtained transformed plant can be used in a conventional breeding scheme to produce more transformed plants with the same characteristics or to introduce the chimeric gene according to the invention in other varieties of the same or related plant species, or in hybrid plants. Seeds obtained from the transformed plants contain the chimeric genes of the invention as a stable genomic insert and are also encompassed by the invention.

The methods and means described herein are believed to be suitable for all plant cells and plants, both dicotyledonous and monocotyledonous plant cells and plants including but not limited to cotton, Brassica vegetables, oilseed rape, wheat, corn or maize, barley, sunflowers, rice, oats, sugarcane, soybean, vegetables (including chicory, lettuce, tomato), tobacco, potato, sugarbeet, papaya, pineapple, mango, Arabidopsis thaliana, but also plants used in horticulture, floriculture or forestry. Especially suited are oil producing plants such as rapeseed (Brassica spp.), flax (Linum usitatissimum), safflower (Carthamus tinctorius), sunflower (Helianthus annuus), maize or corn (Zea mays), soybean (Glycine max), mustard (Brassica spp. and Sinapis alba), crambe(Crambe abyssinica), eruca (Eruca saiva), oil palm (Elaeis guineeis), cottonseed (Gossypium spp.), groundnut (Arachis hypogaea), coconut (Cocus nucifera), castor bean (Ricinus communis), coriander (Coriandrum sativum), squash (Cucurbita maxima), Brazil nut (Bertholletia excelsa) or jojoba (Simmondsia chinensis) gold-of-pleasure (Camelina sativa), purging nut (Jatropha curcas), Echium spp., calendula (Calendula officinalis), olive (Olea europaea), wheat (Triticum spp.), oat (Avena spp.), rye (Secale cereale), rice (Oryza sativa), Lesquerella spp., Cuphea spp., meadow foam (Limnanthes alba), avocado (Persea Americana), hazelnut (Corylus), sesame (Sesamum indicum), safflower (Carthamus tinctorius), tung tree (Aleurites fordii), poppy (Papaver somniferum) tobacco (Nicotiana spp.).

The methods and means described herein can also be used in algae such as Scenedesmus dimorphus, Euglena gracilis, Phaeodactylum tricornutum, Pleurochrysis carterae, Prymnesium parvum, Tetraselmis chui, Tetraselmis suecica, Isochrysis galbana, Nannochloropsis salina, Botryococcus braunii, Dunaliella tertiolecta, Nannochloris spp. or Spirulina spp.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined, may comprise additional DNA regions etc.

The following examples describe the relocalization of mouse FAR and mouse WS to a similar subdomain of the ER and the improved production of the desired wax esters.

Unless stated otherwise in the Examples, all recombinant techniques are carried out according to standard protocols as described in “Sambrook J and Russell DW (eds.) (2001) Molecular Cloning: A Laboratory Manual, 3rd Edition, Cold Spring Harbor Laboratory Press, New York” and in “Ausubel F A, Brent R, Kingston R E, Moore D D, Seidman J G, Smith J A and Struhl K (eds.) (2006) Current Protocols in Molecular Biology. John Wiley & Sons, New York”.

Standard materials and references are described in “Croy RDD (ed.) (1993) Plant Molecular Biology LabFax, BIOS Scientific Publishers Ltd., Oxford and Blackwell Scientific Publications, Oxford” and in “Brown T A, (1998) Molecular Biology LabFax, 2nd Edition, Academic Press, San Diego”. Standard materials and methods for polymerase chain reactions (PCR) can be found in “McPherson M J and Møller S G (2000) PCR (The Basics), BIOS Scientific Publishers Ltd., Oxford” and in “PCR Applications Manual, 3rd Edition (2006), Roche Diagnostics GmbH, Mannheim or www.roche-applied-science.com.

In the description and examples, reference is made to the following sequences:

SEQ ID No. 1: mouse fatty acyl-CoA reductase—nucleotide sequence

SEQ ID No. 2: mouse fatty acyl-CoA reductase—amino acid sequence

SEQ ID No. 3: mouse fatty acyl-CoA reductase tagged with mCherry—nucleotide sequence

SEQ ID No. 4: mouse fatty acyl-CoA reductase tagged with mCherry—amino acid sequence

SEQ ID No. 5: mouse fatty acyl-CoA reductase truncated—nucleotide sequence

SEQ ID No. 6: mouse fatty acyl-CoA reductase truncated—amino acid sequence

SEQ ID No. 7: mouse fatty acyl-CoA reductase truncated tagged with mCherry—nucleotide sequence

SEQ ID No. 8: mouse fatty acyl-CoA reductase truncated tagged with mCherry—amino acid sequence

SEQ ID No. 9: mouse fatty acyl-CoA reductase truncated tagged with mCherry and fused to oleosin—nucleotide sequence

SEQ ID No. 10: mouse fatty-acyl CoA reductase truncated tagged with mCherry and fused to oleosin—amino acid sequence

SEQ ID No. 11: wax ester synthase from mouse—nucleotide sequence

SEQ ID No. 12: wax ester synthase from mouse—amino acid sequence

SEQ ID No. 13: wax ester synthase from mouse tagged with YFP—nucleotide sequence

SEQ ID No. 14: wax ester synthase from mouse tagged with YFP—amino acid sequence

SEQ ID No. 15: wax ester synthase from mouse tagged with YFP fused to oleosin—nucleotide sequence

SEQ ID No. 16: wax ester synthase from mouse tagged with YFP fused to oleosin—amino acid sequence

### EXAMPLES

### Example 1

### Materials and Methods

For all methods sterile pipette tips and reaction tubes were used. All solutions were set up with sterile water (ddH2O).

Hardware/Equipment

ABI PRISM® 3100 Genetic Analyzer Applied Biosystems, Foster, USA

Automatic TLC Sampler 4 Camag, Berlin, Germany

Centrifuge 5810 R Eppendorf AG, Hamburg, Germany

Centrifuge 5415 D Eppendorf AG, Hamburg, Germany

Centrifuge 5417 R Eppendorf AG, Hamburg, Germany

Chromatogramm Immersion Devise III Camag, Berlin, Germany

ColorView II 3.3 MegaPixel CCD Camera Olympus, Hamburg, Germany

Gel-Detection System DIANA Raytest, Straubenhardt, Germany

Gel-Detection System AIDA Raytest, Straubenhardt, Germany

Glass beads Roth, Karlsruhe, Germany

Gold particles BioRad, Hercules, USA

Mastercycler personal Eppendorf, Wesseling-Berzdorf, Germany

Olympus BX51 Olympus, Hamburg, Germany

Olympus U-RFL-T Olympus, Hamburg, Germany

PDS1000/He Biolistic Particle Delivery System BioRad, Hercules, USA

TLC Plate heater III Camag, Berlin, Germany

TLC Silica Gel 60 20×20 cm Merck, Darmstadt, Germany

Ultrospec 1100pro Amersham Biosciences, Freiburg, Germany

3 MM-paper Whatman, Madistone, Kent, UK

6890 Series GC System Agilent, Waldbronn, Germany

Computer programs: analysis wincat

Chemicals

Unless otherwise indicated all chemicals were obtained from either, Sigma-Aldrich (Munich, Germany) or Roth (Karlsruhe, Germany). Organic solvents such as n-hexane, methanol, ethanol, acetonitrile or diethyl ether were obtained from Acros, Geel, Netherlands or Baker, Griesheim, Germany.

Fatty Acid and Lipid Standards

Internal standard for fatty acid quantification:

1,2,3-triheptadecanoyl glyceryl Sigma-Aldrich (München,Germany)

Standards for thin layer chromatography:

Triacylglycerol/Diacylglycerol-mixture Sigma-Aldrich (München,Germany)

Palmitate Sigma-Aldrich (München,Germany)

Palmityl alcohol Sigma-Aldrich (München,Germany)

Palmityl palmitate Sigma-Aldrich (München,Germany)

Sterol esters kindly provided by Sabine Freitag and Dr. Cornelia Göbel

Enzymes

Restriction Enzymes

The restriction enzymes XhoI, EcoRI, BamHI, SalI, HindIII, NotI and SacII were purchased from MBI Fermentas (St. Leon-Rot, Germany) and the restriction enzyme Bsp14071 was obtained from NewEngland Biolabs (Ipswich, UK). All enzymes were used according to the manufacturer's protocol.

Other Enzymes

T4-DNA-Ligase Fermentas, St.-Leon Roth, Germany

Phusion™ DNA-Polymerase Finnzymes, Espoo, Finland

Gateway®LR Clonase™ II Enzym Mix Invitrogen, Karlsruhe, Germany

Kits and Systems

Macherey-Nagel NucleoSpin®Plasmid Macherey-Nagel, Duren, Germany

NucleoSpin® Extract Kit Macherey-Nagel, Düren, Germany

CompactPrep® Plasmid Midi Core Kit Qiagen, Hilden, Germany

ABI PRISM® BigDye™ Terminator Foster City, Calif., USA

Cycle Applied Biosystems

Sequencing Ready Reaction Kit v1.1 Invitrogen, Karlsruhe, Germany

ProQuest™ Two-Hybrid System Invitrogen, Karlsruhe, Germany

Oligonucleotides (Primers)

Primer for Cloning Primer Sequences

FAR2-EcoRI-for
5′-GGATGCGAATTCATGAGCATGATCGCAGCTTTC-3′
FAR2-XhoI-rev
5′-GGATGCCTCGAGTTAGACCTTCAAAGTACTGGA-3′
FAR2c-XhoI-rev
5′-GGATGCCTCGAGTTAATGTATGTTACGCAAACGCC-3′
FAR2g.for.EcoRI
5′-GGCCATGCGAATTCATGATACATTATCTTTTTAATACTG-3′
FAR2g.rev.XhoI
5′-GGCCATGCCTCGAGTTAGACCTTCAAAGTACTGGAGGC-3′
FAR1-EcoRI-for
5′-GGATGCGAATTCATGGTTTCCATCCCAGAGTAC-3′
FAR1-rev-XhoI
5′-ATGCCTCGAGTTAGTATCGCATTGTGGAAGAGGC-3′
FAR1c-XhoI-rev
5′-GGATGCCTCGAGTTATCTGATGTTGCGAAGCTTGTT-3′
mCherry-for-BamHI
5′-ATGCGGATCCATGGTGAGCAAGGGCGAGGAGGAT-3′
mCherry-rev-EcoRI-stop
5′-ATGCGAATTCCTTGTACAGCTCGTCCATGCCGCC-3′

Primer for Sequencing Primer Sequences

M13 uni
5′-CCCAGTCACGACGTTGTAAAACG-3′
M13rev
5′-AGCGGATAACAATTTCACACAGG-3′
mCherry-mitte.for,
5′-CACTACGACGCTGAGGTCAAGA-3′
FAR2-mitte-for
5′-GAGGTCTATTAAGGCTACTC-3′
FAR2-mitte-rev
5′-GAGTAGCCTTAATAGACCTC-3′
MmWS-mitte-for
5′-CTATGGACTTCTTGCTTAC-3′
MmWS-mitte-for
5′-GTAAGCAAGAAGTCCATAG-3′

Plasmids

For plant constructs:

pUC18-ENTRY2 AmpR, modified pUC18 vector containing attL1 and attL2 Gateway cloning sites (modified and provided by Dr. Ellen Hornung;.Hoffmann et al., 2007)

pCAMBIA3300 KanR in bacteria, Glufosinate (BastaR) in plants under control of the CaMV 35S-promotor. The vector includes two recognition sites, namely auR1 and attR2, which enables insertion of the DNA fragment due to homologous recombination. It is inserted in reading direction of the 35S-promotor. (Modified and provided by Dr. Ellen Hornung)

Yeast expression vector:

pESC-URA AmpR (Stratagene, Amsterdam, Netherlands)

Organisms

Bacteria strains

Escherichia coli XL1blue Genotype: endA1, hsdR17, supE44, thi-1, rec A1, gyrA96, relA1, lac, [F′, proAB, laclqZΔ15, Tn10(tetR)] (Stratagene, Amsterdam, Netherlands)

Yeast Strains

Saccharomyces cerevisiae INVSc1 Genotype: his3 Δ1/his3 Δ1, leu2/leu2, trp1-289/trp1-289, ura3-52/ura3-52 (Invitrogen, Karlsruhe, Deutschland)

Saccharomyces cerevisiae W303 H1246 Genotype: MATα ADE2-1 can1-100 ura 3-1 are 1-Δ::HIS3 are2-Δ::LEU2, dgal-Δ::KanMX4 Irol-Δ::TRP1(Sten Styme (Scandinavian Biotechnology Research, Alnarp, Sweden)

Saccharomyces cerevisiae MaV103 Genotype: MATα, leu2-3,112, trp1-901,his3 α200, ade2-101, gal4Δ, ga180Δ, SPAL10::URA3, GAL1::lacZ, HIS3UAS GAL1::HIS3@LYS2, can1R, cyh2R) (Invitrogen, Karlsruhe, Germany)

Plants

Allium cepa (Onion)

Culture media

For producing solid media 1.5% Micro-Agar (Duchefa, Haarlem, Netherlands) was added to bacteria media; 2% were added to yeast media. All media were autoclaved after solving.

Medium for E. coli

LB (Luria and Bertani) medium Contains 1% (w/v) peptone, 0.5% (w/v) yeast extract and 1% (w/v) NaCl. pH value was adjusted with NaOH to pH 7.

Yeast Media

SD medium 5 g/L ammonium sulfate, 1.7 g/l YNB w/o ammonium sulfate; add sterile water to 1 L

Dropout powder:

Ingredients Amount (g)
L-Arginine (HCl) 2

L-Histidine (HCl) 2

L-Isoleucine 2

L-Lysine (HC1) 2

L-Methionine 2

L-Phenylalanine 2

L-Serine 2

L-Threonine 2

L-Tyrosine 2

L-Valine 9

The different substances were mixed and ground to a fine powder. Depending on the selection of the vector, either uracil, leucine and/or tryptophan (2 g each) were added separately to the powder. YPD medium 1% (w/v) yeast extract, 2% (w/v) peptone and 2% (w/v) glucose.

Media Supplements for Selection

antibiotics solved in stock solution end concentration
kanamycin ddH2O 50 mg/ml 25 μg/l
carbenicillin ddH2O 100 mg/ml 100 μg/l
gentamicin ddH2O 50 mg/ml 50 μg/l

Transformation of E. coli

For transformation of E. coli the strain XLlblue was used. This strain is known to be suited for heat shock transformation as performed.

Preparation of Competent Cells

The preparation of competent E. coli cells was accomplished after the method established by (Inoue et al., 1990).

Transformation of Competent E. coli Cells

For transformation competent cells were gently thawn on ice. Then 100 μl of cell suspension were added to either a 10 μl ligation reaction or to 1 μl of plasmid DNA for retransformation of vector constructs. The reaction were incubated on ice for 30 minutes and further put to 42° C. for 30 seconds. Afterwards, cells were placed on ice again for roughly 3 minutes. 900 μl of LB medium were added to the samples and those were incubated at 37° C. for one hour while shaking. After that, cells were spun down at 3000 rpm, supernatant was discarded and the pellet was resuspended. Cells were plated on solid LB medium containing the specific antibiotic for selection and incubated overnight at 37° C.

Transformation of S. cerevisiae

2 ml of YPD medium were inoculated with colonies of either the INVSc1 or the H1246 yeast strain. The pre-cultures were incubated overnight at 30° C. while shaking at 200 rpm. The next day, 50 ml of YDP-medium were inoculated with the pre-culture to an optical density at 600 nm (OD600) of 0.15 and grown at 30° C. to an OD600 of 0.6-0.7. Cells were sedimented by centrifugation (700 g at room temperature), the cells were washed with 20 ml 1x TE-buffer (100 mM Tris-HCl, 10 mM EDTA, pH 8), afterwards resuspended in 1 ml 1x LiAc/1x TE and incubated for 10 minutes at room temperature. 5 μl of Plasmid-DNA were mixed with 100 μl of prepared cells and 700 μl PEG 4000/1xLiAc-mix and incubated for 30 minutes at 30° C. In the following, 88 μl DMSO were added and cells were then incubated at 42° C. for 15 minutes. Afterwards, cells were centrifuged, the supernatant was discarded and the cells were washed in 500 μl 1x TE-buffer. The remaining yeast cells were resuspended in 100 μl 1x TE-buffer and selected on SD plates.

Yeast Expression Cultures

Yeast expression cultures were performed in order to purify lipids or fatty acids. Therefore, transformed yeast colonies were resuspended in 2 ml SD medium with the corresponding dropout and incubated over night at 30° C. while shaking at 200 rpm. Then, the OD600 was measured and the amount (in ml) of cell-culture was calculated to inoculate 20 ml expression culture to an OD600 of 0.3. The expression cultures were incubated while shaking at 200 rpm for ca. 48 hours at 30° C. Afterwards, similar OD600-units of expression cultures were harvested for lipid analysis.

Transformation of A. cepa (Onion)

For transient transformation fresh onions were used.

Gold Particle Precipitation

Aliquots containing 25 μl of gold suspension (50 mg/ml; solved in ethanol) were mixed vigorously with 3-8 μg of each plasmid-DNA (pCAMBIA3300 constructs and/or marker constructs), 55 μl of ddH2O, 50 μl CaCl2 (2.5 M) and 20 μl spermidin (0.1 mM). The gold-DNA mixture was precipitated via centrifugation at maximal speed for 10 seconds and washed three times with 100 μl 100% ethanol. Finally, the precipitate was resuspended in 30 μl 100% ethanol. The plasmid-DNA was then ready for transforming onion epidermal cells.

Transient Transformation of Onion Epidermal Cells Via Gold Particle Bombardment

Onion cells were transiently transformed using the PDS 1000/He Biolistic Particle Delivery System (BioRad, Hercules, USA). First, 5 μl of plasmid-coated gold suspension (2.13.1) were placed centered on the macrocarrier and allowed to dry at room temperature. Further, the rupture disk, the macrocarrier (with gold particles) and the stopping screen were set into their correct position and the sliced onion was placed on the target plate. Then, a vacuum was set up in the chamber of the Particle Delivery System and high pressure was applied until the rupture disk broke. Due to this, the macrocarrier containing the gold particles was pressed down onto and the stopping screen, scattering the now accelerated gold particles, which then hit the onions surface with high speed. The transformed onion pieces were incubated overnight at room temperature and under high humidity to avoid desiccation of the onion. Microscopic evaluation was performed the next day.

Fluorescence Microscopy

Transiently transformed onion epidermal cells (see 2.13.2) were evaluated using the BX51 fluorescence microscope (Olympus, Hamburg, Germany). Therefore the epidermis of the onion piece was carefully ripped of using forceps and placed on an object plate with water before a cover slip was placed on top. Depending on the used fluorescent marker (mCherry, YFP and CFP) different UV-filters were required to detect the marked cells with 20× or 40× magnification. Pictures were taken using the ColorView II 3.3 MegaPixel CCD Camera (Olympus, Hamburg, Germany).

Lipid Analysis

Lipid Extraction from Yeast and Separation of Lipid Classes

All following extraction procedures were carried out in glass reaction tubes. For lipids analysis yeast expression has been carried out with 20 ml cultures. A certain amount of cells (measured by OD600 units) out of 20 ml yeast expression culture (see 2.12.1) were harvested by centrifugation at 700 g for 3 min at room temperature. The supernatant was discarded completely and the cells were disrupted in 1 ml methanol and glass beads (1.7-2 mm, Roth, Karlsruhe, Germany) by vigorously mixing for 20 minutes. The samples were incubated for 15 minutes at room temperature. Further, 1 ml Chloroform was added and the samples were again mixed vigorously for 20 minutes. After that, the samples were centrifuged and the supernatant (equivalent with the lipid extract) was transferred into a new glass reaction tube and was evaporated under nitrogen. The remaining cell debris was resuspended in 2 ml hexane:diethyl ether:formic acid (65:35:1 v/v/v), mixed well and incubated at room temperature for 15 minutes. The samples were centrifuged and the supernatant was fused with the already evaporating lipid extract. The dried lipid extracts were dissolved in 100 μl chloroform and transferred into glass inlays. The solvent was evaporated and finally the lipid residues were solved in 50 μl of chloroform to ensure consistent solvent volume. The lipid extracts could then be analyzed by thin layer chromatography.

Thin Layer Chromatography (TLC)

Portions of 25 μl of the lipid extracts (2.20.1) were spotted on a silica gel TLC plate using the Automatic TLC Sampler 4 (Camag, Berlin, Germany). Lipid standards (hexadecanol, palmitic acid, palmitoyl palmitate, 1,2,3-triheptadecanoyl glyceryl, sterol esters, diacylglycerol, were dissolved in chloroform at a final concentration of 1 mg/ml and 2 μl aliquots were spotted on the TLC plates adjacent to the lipid extracts. TLC plates were developed in hexane:diethyl ether:formic acid (90:10:1, v/v/v) when the formation of wax esters were analyzed and hexane:diethylether:formic acid (65:35:1 v/v/v) when fatty alcohols were analyzed. After development, lipids were visualized on TLC by dipping the plate in a copper sulfate solution (10 g CuSO4×5 H2O; 0.8% H3PO4 in 100 ml H2O) and charring by 180° C.

Fatty Acid Analysis

Acidic Hydrolysis and Extraction of Fatty Acid Methyl Esters (FAMEs)

For fatty acid analysis yeast expression has been carried out with 20 ml cultures. A certain amount of cells (measured by OD600 units) out of 20 ml yeast expression culture were harvested by centrifugation at 700 g for 3 min at room temperature. Fatty acid methyl esters (FAMEs) were obtained by methylation of yeast cell sediments with methanol containing 2.75% (v/v) sulfuric acid and 2% (v/v) dimethoxypropane at 80° C. for 1 h. As internal standard 1,2,3 triheptadecanoyl glycerol (stock solution 1 mg/ml) were added to each sample. The reaction was neutralized by adding 0.1 ml NaCl (5 M) and FAMEs were extracted in 2 ml of n-hexane, dried under N2, finally resolved in GC plastic inlays in 10 μl acetonitrile and analyzed by gas chromatography (GC).

Identification of FAMEs by GC with Flame Ionization Detection

The GC analysis was performed with an Agilent GC 6890 system coupled with a flameionization detector equipped with a capillary 122-2332 DB-23 column (30 m×0.32 mm; 0.5 μm coating thickness; Agilent). Helium was used as carrier gas (1 ml min-1). Samples were injected at 220° C. The temperature gradient was 150° C. for 1 min, 150 to 200° C. at 15° C. min-1, 200 to 250° C. at 2° C. min-1, and 250° C. for 10 min. Data were processed using the HP ChemStation Rev. A09.03. FAMEs were identified by comparison with appropriate reference substances.

### Example 2

### Plant and Yeast Expression Constructs Used Herein.

To perform retargeting experiments, truncated versions of MmFAR1 were generated to determine which domains are essential for peroxisomal localization and which further domains can retarget the MmFAR enzymes from the peroxisomes to the cytosol or ER.

mCherry is a red fluorescent protein and its excitation and emission maxima are 587 nm and 610 nm, respectively. Expression of fusion proteins with mCherry enables to determine their localization in vivo by fluorescence microscopy. mCherry was cloned by PCR amplification into the BamHI and EcoRI sites (5′ and 3′ of mCherry, respectively) of the pUC18-ENTRY or the pESC-URA plasmids. The full length and the truncated open reading frames (ORFs) of MmFAR1 were modified using the Phusion Polymerase (Finnzymes, Espoo, Finland) and a standard PCR protocol with the respective primers listed in Materials and methods, introducing an EcoRI restriction site at the 5′ prime end and a XhoI restriction site at the 3′ prime end of the respective PCR products. The PCR products were then moved as EcoRI/XhoI fragments in frame to the already cloned Cherry either into the Gateway donor-vector pUC18-ENTRY or into the yeast expression vector pESC-URA, yielding the constructs mCherry-MmFAR1, mCherry-MmFAR1c and in pUC18-ENTRY as well as in pESC-URA (FIG. 7 and nucleotide sequences in the sequence listing).

Arabidopsis thaliana oleosin proteins are proteins which are targeted to oil bodies via the ER (Huang, 1992). The conformation of oleosins is unique with ER membrane proteins: their membrane-integrated hydrophobic domain is flanked by N- and C terminal domains located on the outer microsomal surface of the ER (Abell et al., 1997). This membrane topology on the ER allows the fusion of further proteins either to the N or the C-terminus. A. thaliana oleosin 3 fusion constructs were established by amplification of oleosin 3 from A. thaliana cDNA with the respective primers, introducing a SalI restriction site at the 5′ prime end and a BamHI restriction site at the 3′ prime end of the respective PCR product. The amplicon of oleosin 3 was partially restricted (within the oleosin 3 sequence an additional BamHI restriction site is localized) and moved as N-terminal fusion to the different mCherry-FAR-constructs in pUC 18-ENTRY and pESC-URA, yielding Oleo3-mCherry-MmFAR1 or Oleo3-mCherry-MmFAR1c (FIG. 7 and nucleotide sequences in the sequence listing).

The MmWS was cloned as oleosin 3-YFP fusion construct, named Oleo3-YFP-WS and cloned via SalI and XhoI restriction sites into pUC18-ENTRY vector like the MmFAR constructs (FIG. 7 and nucleotide sequences in the sequence listing).

### Example 3

### Subcellular Localization of MmFar1 and truncated MmFar1c and Fusion Proteins to Oleosin by Transient Expression in Onion Epidermis Cells.

The fusion- or the truncated mCherry-MmFAR1 constructs, arranged in the pUC18-ENTRY vector, were transferred into the plant expression vector pCAMBIA3300 by clonase reaction. After selection, the positive clones were precipitated on gold particles along with a peroxisomal marker (MDH). Those particles were then shot in onion cells in order of transient transformation. After incubation over night the transformed onion cells were examined via fluorescent microscopy.

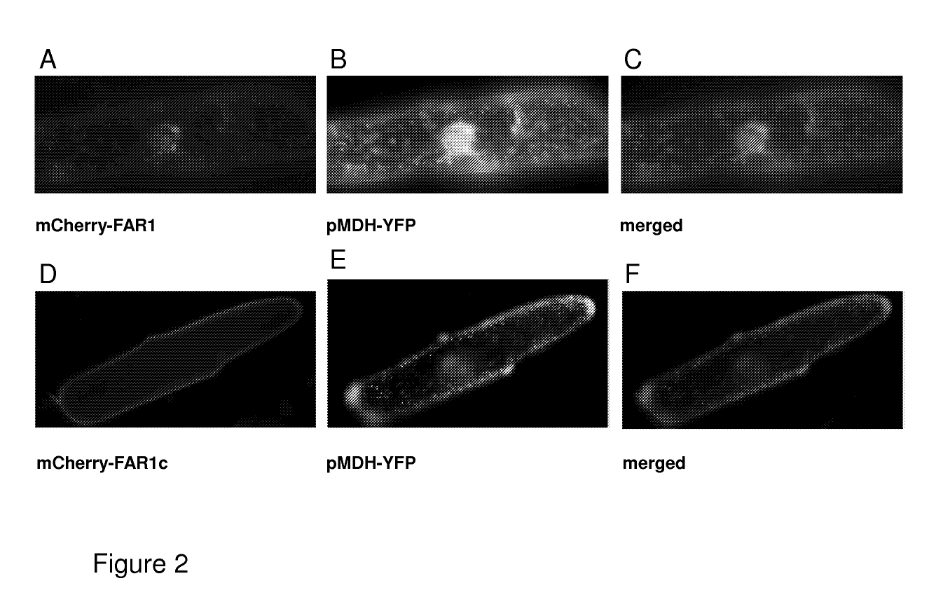

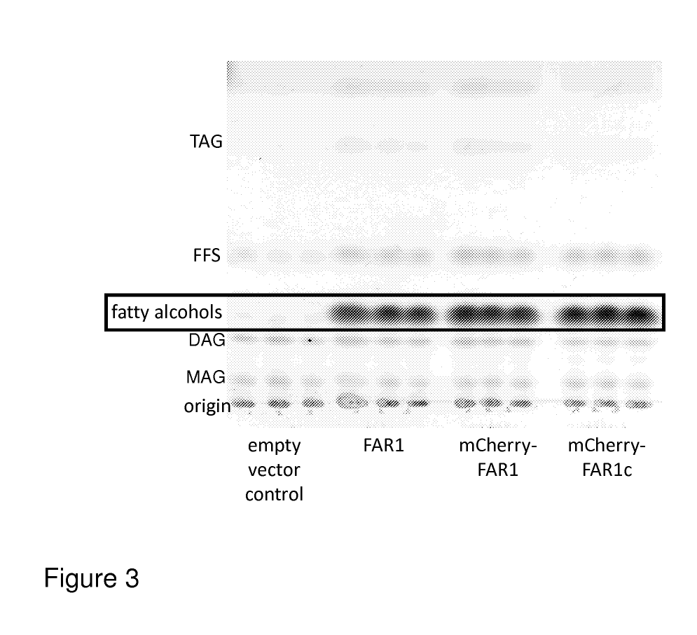

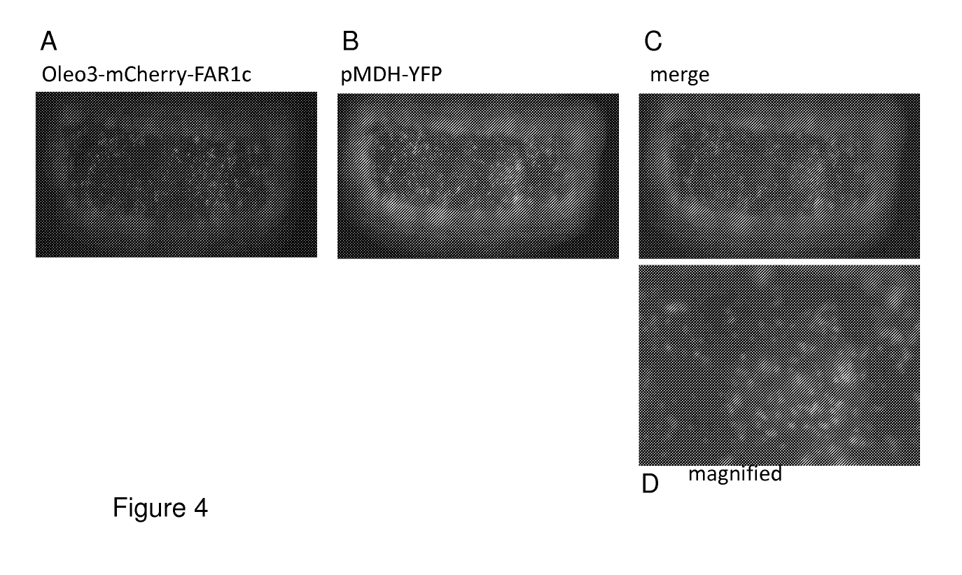

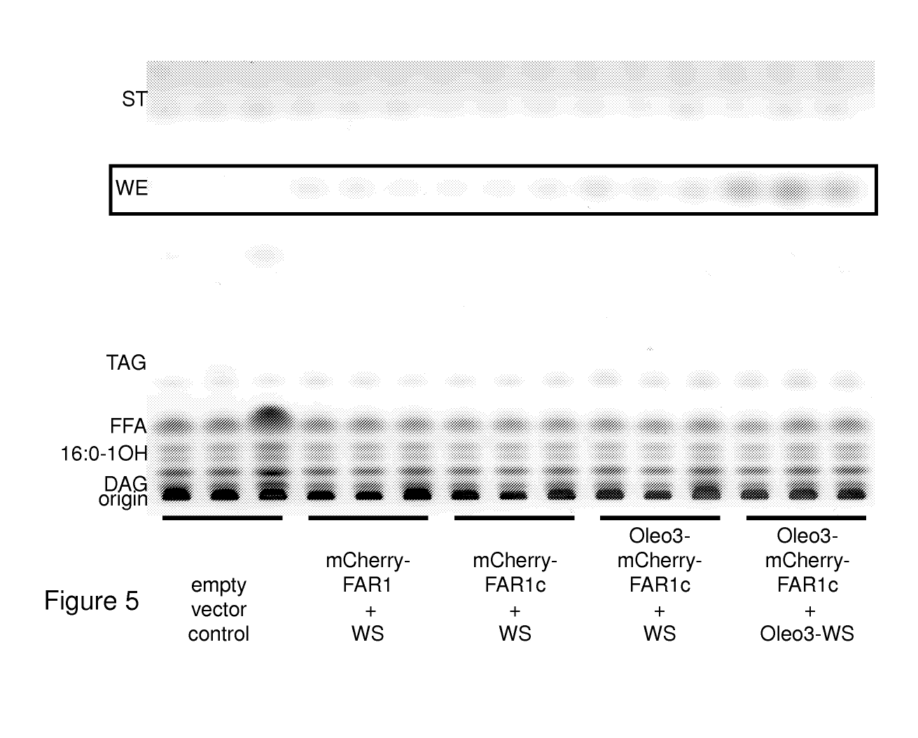

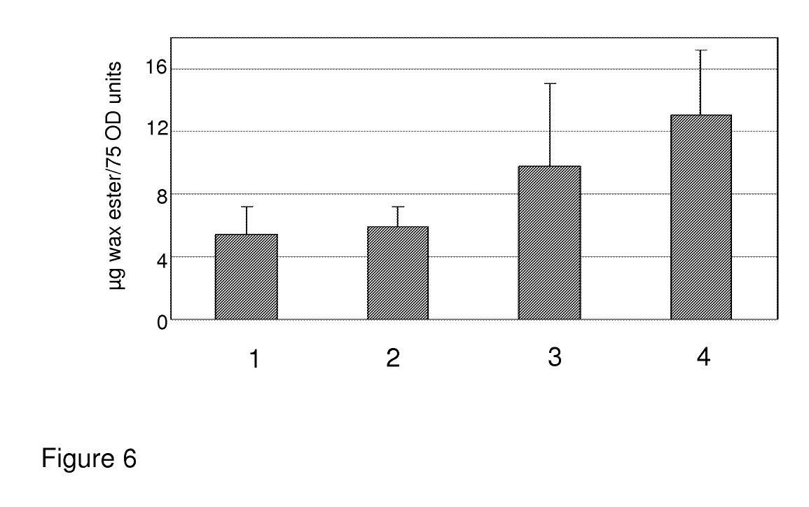

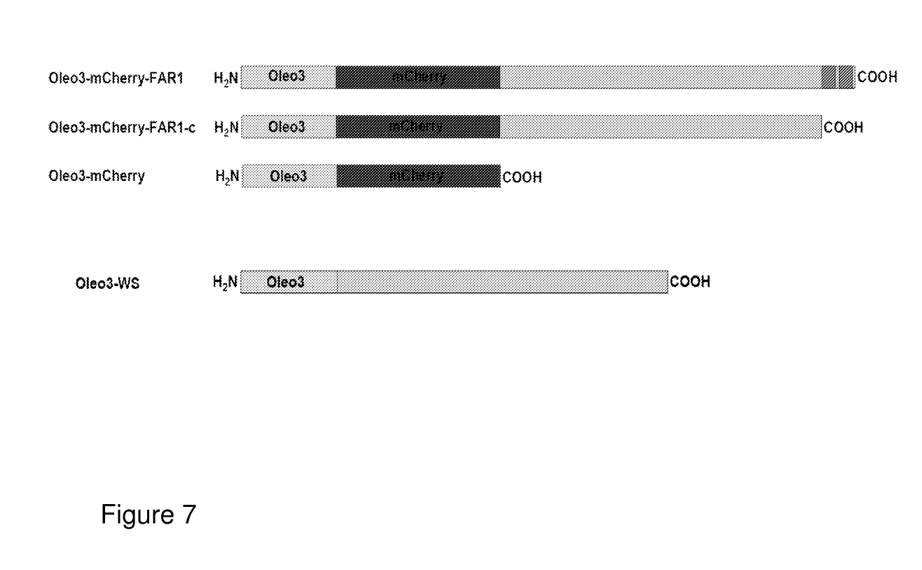

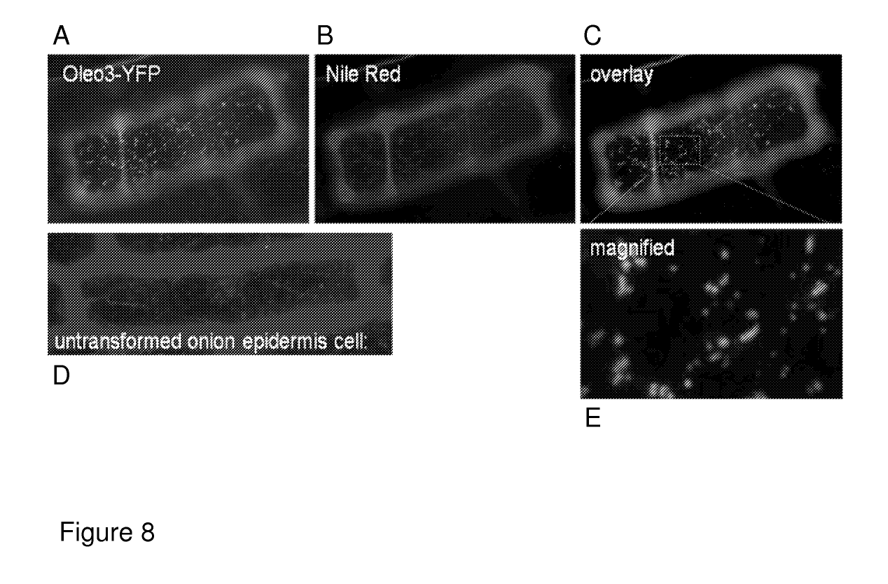

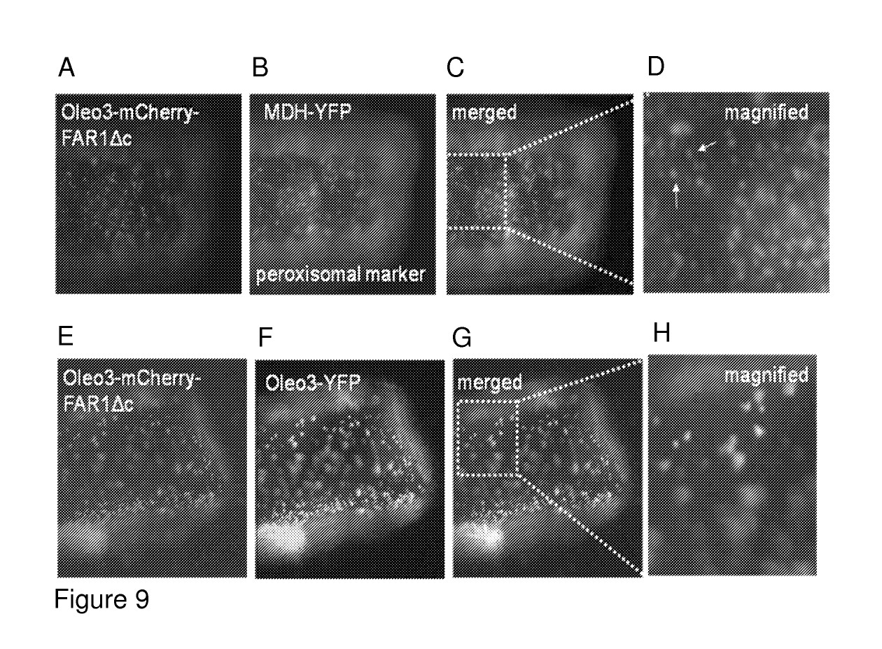

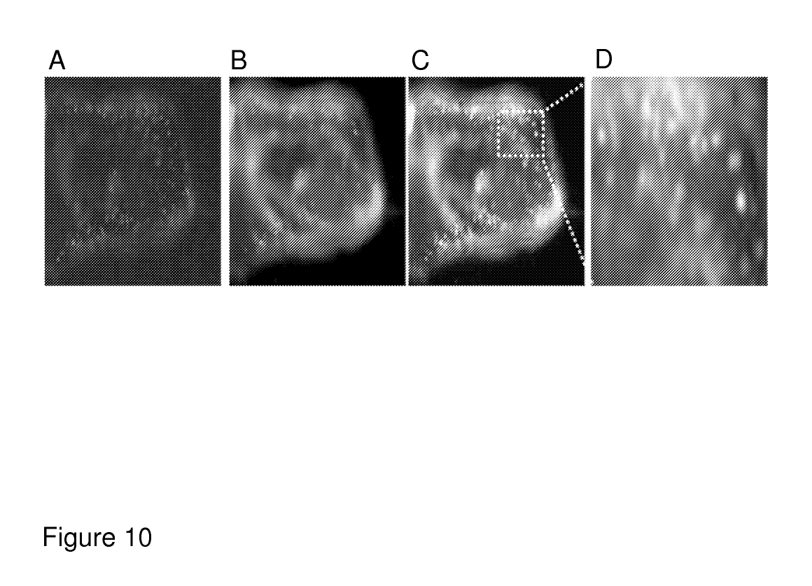

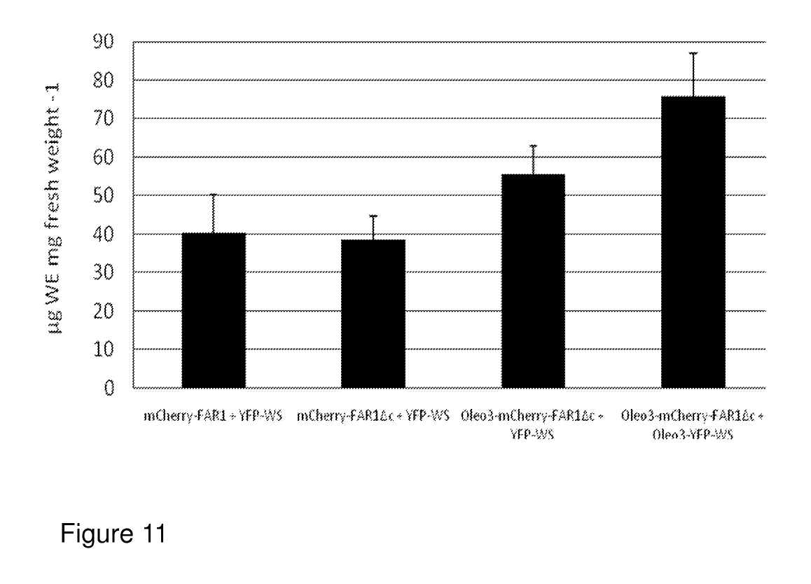

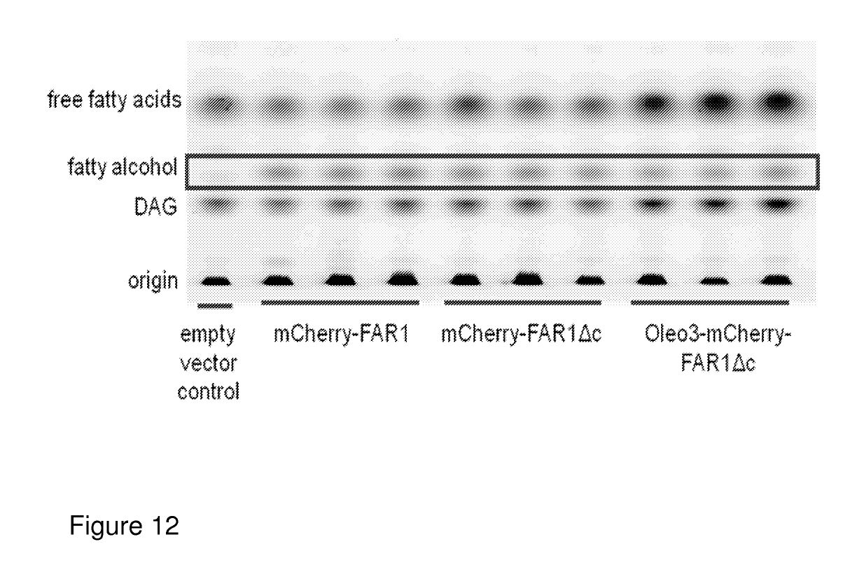

FIG. 2 shows the localization of the diverse MmFAR1 constructs tagged to mCherry. Images A of FIG. 2 shows onion epidermis cells expressing the mCherry-MmFAR1 construct. The fluorescence of mCherry-MmFAR1 was detected in the cytoplasm as small puncta that co-localized with the peroxisomal marker protein MDH (FIG. 2 B). observation corresponds to the described peroxisomal localization of MmFAR1 in animal cells reported by Cheng and Russel 2004.

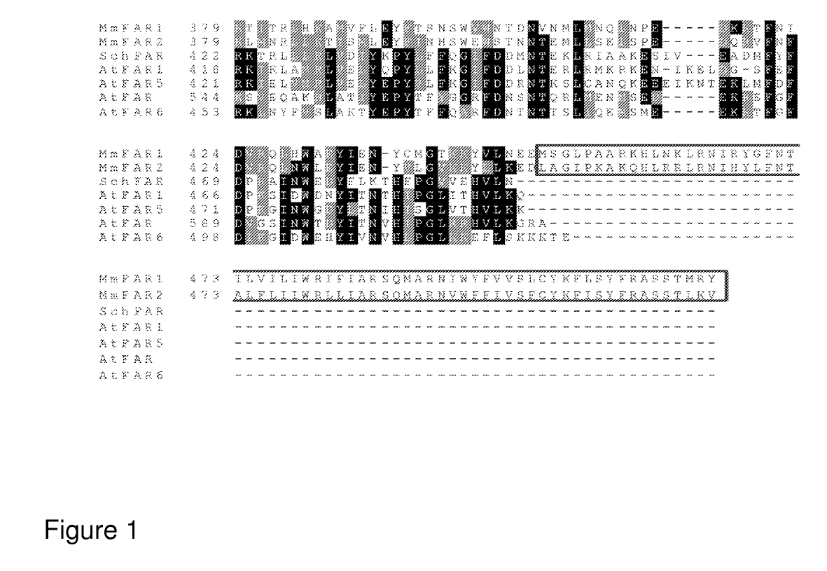

As shown for MmFAR1c, the truncation of the C-terminal transmembrane domains of MmFAR1 (see FIG. 1) leads to the re-localization of the MmFAR1c protein: from peroxisomal to a presumable cytosolic localization (FIG. 2 D). Co-expression of mCherry-MmFAR1c with the peroxisomal marker MDH (FIG. 2 E).

The AtOleosin 3 protein is known to be found in lipid bodies in Arabidopsis thaliana. By fusing mCherry-MmFAR1 and mCherry-MmFAR1c to AtOleosin 3 it could be examined, if oleosin 3 fusion were localized at the lipid bodies resulting in a retargeting of the associated MmFAR proteins as well. The retargeting towards lipid bodies is of high interest because lipid bodies evolve from the ER, where the MmFAR enzymes preferably should target to.

In FIG. 4 the images A-C show the oleosin 3-mCherry-MmFAR1 construct coexpressed in onion cells with the peroxisomal marker MDH. In image A, as well as in image B fluorescent spots are visible. By merging these two images it becomes apparent that, although in both images spots are recognizable, these spots show totally different patterns which cannot be explained by the time difference while changing the filters.

In FIG. 4D this different pattern can be observed more in detail in image A. Due to the difference in pattern it is taught, that the Oleo3-mCherry-MmFAR1 construct does not localize at the peroxisomes. Further, although the construct was not coexpressed with a lipid body marker, it is highly presumable that the constructs localizes at lipid bodies. One the one hand due to the dropwise distribution throughout the whole cell, on the other hand because oleosin 3 is known to localize at lipid bodies.

Similar results were obtained for Oleo3-mCherry-MmFAR1c, indicating this protein is localized at the lipid bodies as well.

To investigate whether onion epidermis cells indeed contain lipid bodies or comparable structures, onion epidermis cells, expressing transiently YFP-Oleosin3 or without expression of any transgene, were stained with Nile red. Nile red is known to color oil bodies or lipid containing structures. As shown in FIG. 8, dot like structures are visible within the untransformed onion epidermis cell.

Co-localization tests with YFP-Oleo3 show, that the localization of YFP-Oleo3 is visible as green fluorescent spots which merge with the Nile red stained droplets. Because Oleosin is known as lipid body marker, it therefore appears that, first, onion epidermal cells contain lipid bodies, or lipid body-like structures and second, that Oleo3-mCherry-FAR1c is localized at lipid bodies in onion epidermal cells, because it co-localizes with Oleo3-YFP, which on the other hand co-localizes with Nile red stained structures.

FIG. 9 shows coexpression of Oleo3-mCherry-MmFAR1c with the peroxisomal marker MDH (A-D), and with YFP-Oleosin as oil body marker (E-H). As already mentioned, the fluorescent pattern does not merge with the fluorescent pattern of the Oleo3-mCherry-MmFAR1c-fusion protein although both appear as fluorescent spots.

Co-localization of Oleosin-tagged MmFAR1c with YFP-Oleosin as oil body marker shows clearly that Oleosin-tagged MmFAR1c localizes at lipid bodies.

As shown above, it is possible to retarget mCherry-FAR1c from cytosolic localization towards the oil bodies by connecting mCherry-MmFAR1c to AtOleosin3. Additionally YFP-WS was fused to At-Oleosin in order to achieve a co-localization at micro-domain level. To test the co-localization of Oleo3-mCherry-FAR1c with Oleo3-YFP-WS, both constructs were co-bombardedinto onion epidermis cells and the fluorescent patterns were compared. As shown in FIG. 10, it is visible that both constructs localize in droplet-like structures as observed before. By merging the images of Oleosin3-mCherry-MmFAR1c and Oleo3-YFP-WS it becomes apparent that these spots show in most cases the same patterns and merges well.

### Example 4

### Enzymatic Activity of MmFar1, MmFar1 and Mm Far1c and oleo-FAR1c Tagged with mCherry in Yeast

After it was demonstrated by fluorescent microscopy that the truncated MmFAR1c constructs were no longer localized at the peroxisomes but in the cytosol, it was tested, if the activity of the wild type version and the truncated version are equal, or if a decrease/increase is detectable. It was also tested, how activity behaves by tagging the MmFAR enzyme to AtOleosin3. For this purpose, yeast (H1246) cells were transformed with the pESC-URA vectors containing the appropriate construct. After expression for 48 hours (FIG. 3) or 72 hours (FIG. 12), the lipids were harvested and separated by TLC.

The results demonstrated that expression of yeast of FAR1 (wt), FAR1 tagged with mCherry, Far1c tagged with mCherry, and FAR1c tagged with mCherry and oleosin all resulted in similar amounts of fatty alcohols being produced. Accordingly, neither the addition of mCherry tag, nor the truncation, nor the addition of oleosin influence the enzymatic activity in yeast.

### Example 5

### Co-Expression of MmFar1 and WS in Yeast.

The wax biosynthetic pathway was reconstituted in cultured H1246 (yeast) cells by coexpressing cDNAs encoding unmodified mCherry-MmFAR1 or modified mCherry-MmFAR1c, Oleo3-mCherry-MmFAR1c with the MmWS, respectively. Additionally, co-expression of Oleo3-mCherry-FAR1c with Oleo3-YFP-WS was tested. The extracted lipids were then separated with the developing solvent hexane:diethyl ether:formic acid (90:10:1), which resolves mainly wax esters much better. This experiment was performed and the resulting TLC can be seen in FIG. 5. To identify the separated bands in the TLC (A), the following standards were used:sterols, TAG-mixture, 16:0 fatty acids, 16:0-OH fatty alcohols and wax esters. The tested samples were taken from yeasts transformed with an empty vector (control), or transformed with mCherry-MmFAR1+MmWS (1), mCherry-MmFAR1c+MmWS (2), Oleo3mCherry-MmFAR1c+MmWS (3) or Oleo3mCherry-MmFAR1c+Oleo3YFP-MmWS (4) (three samples each).

The interesting aspect on this plate is the amount of wax esters (marked with a red box). There are no bands detectable in the control samples. Looking at the mCherry-MmFAR1+MmWS (1) samples, all three of them show weak bands in height of the WE running in the standard. Hence, this indicates a production of wax esters, which further as well indicates that the MmFAR1 must be active as well. MmFAR1 provides the 16:0-OH needed to produce WE. If MmFAR1 would not be active no wax esters could be produced as can be seen in the control samples. The samples taken from yeast cells transformed with mCherry-MmFAR1c+MmWS (2) show wax esters in an apparently equal amount compared with the mCherry-MmFAR1+MmWS samples. In the three Oleo3mCherry-MmFAR1c+MmWS (3) samples the WE bands are visible as well, but clearly brighter than in the samples from (1) and (2). Oleo3mCherry-MmFAR1c+Oleo3YFP-MmWS (4) show even brighter bands, indicating an extremely high amount of wax esters produced in those yeast cells. To show the differences in wax ester production of each construct more in detail, a bar chart (FIG. 6) was generated. The intensity of each WE band was measured and further, the average value of every construct was calculated. Next the histogram was produced, including the standard deviation. It can be observed, that the amount of wax esters produced in yeast containing mCherry-MmFAR1+MmWS (1) and mCherry-MmFAR1c+MmWS (2) are nearly similar, whereas the amount of wax esters almost doubles in cells transformed with Oleo3mCherry-MmFAR1c+MmWS (3). Cells expressing Oleo3mCherry-MmFAR1c+Oleo3YFP-MmWS (4) show an increase in wax esters of nearly threefold.

The amounts of wax esters produced in yeast were also analyzed by GC-MS (See FIG. 11). No wax esters could be detected in the empty vector control (data not shown). After expression of mCherry-FAR1+YFP-WS, mCherry-FAR1c+YFP-WS, Oleo3-mCherry-FAR1c+YFP-WS or Oleo3-mCherry-FAR1c+Oleo3-YFP-WS wax esters were produced and identified by standards. While the wax ester amount after expression of mCherry-FAR1+YFP-WS and mCherry-FAR1c+YFP-WS seemed to be very similar, an increase of wax ester production and accumulation is observable with expression of Oleo3-mCherry-FAR1c+YFP-WS and even more with expression of Oleo3-mCherry-FAR1c+Oleo3-YFP-WS.

These results demonstrate that mCherry-MmFAR1, mCherry-MmFAR1c and Oleo3mCherry-MmFAR1c are active in yeast as they provide the substrate needed by the WS to produce wax esters. In addition, the results show that there is no significant difference in activity between MmFAR1 and the truncated MmFAR1c construct, which lacks the last two putative transmembrane domains. Finally, the results show that the activity can be increased by tagging MmFAR1c to AtOleosin3 and that activity can be further amplified by tagging MmFAR1c and MmWS to AtOleosin3.

### Example 6

### Co-expression in Plants of MmFar1-oleosin, MmFar1c-oleosin and MmWS Oleosin.

To introduce a wax ester synthase biosynthetic pathway in plants, the following chimeric genes are constructed using conventional techniques comprising the following DNA fragments in order:

MmFar1-oleosin
a seed-specific promoter such as a promoter of the napin gene (Stalberg K., Ellerstrom M., Josefsson L. and Rask L. (1993). Deletion analysis of a 2S seed storage protein promoter of Brassica napus in transgenic tobacco. Plant Molecular Biology, 23, 671-683) or the USP promoter from Vicia faba described in DE10211617
a DNA fragment encoding an oleosin such as oleosin 3 (SEQ ID No 10 from amino acid 1 to amino acid 143) in, fused in the reading frame with
a DNA region encoding FAR1 from mouse (SEQ ID 1) or a DNA region encoding FAR1 from mouse tagged with mCherry (SEQ ID No 4)
a 3′ transcription termination and polyadenylation region such as the 3′ end of the nopaline synthase gene (3′nos: sequence including the 3′ untranslated region of the nopaline synthase gene from the T-DNA of pTiT37 of Agrobacterium tumefaciens (Depicker et al., 1982, Journal of Molecular and Applied Genetics, 1, 561-573).

MmFar1c-oleosin
a seed-specific promoter such as a promoter of the napin gene or the USP promoter from Vicia faba described in DE10211617
a DNA fragment encoding an oleosin such as oleosin 3 (SEQ ID No 10 from amino acid 1 to amino acid 143) in, fused in the reading frame with
a DNA region encoding truncated FAR1 from mouse (SEQ ID 6) or a DNA region encoding truncated FAR1 from mouse tagged with mCherry (SEQ ID No 8)
a 3′ transcription termination and polyadenylation region from the 3′ end of the nopaline synthase gene (3′nos: sequence including the 3′ untranslated region of the nopaline synthase gene from the T-DNA of pTiT37 of Agrobacterium tumefaciens (Depicker et al., 1982, Journal of Molecular and Applied Genetics, 1, 561-573).

MmWS-oleosin
a seed-specific promoter such as a promoter of the napin gene or the USP promoter from Vicia faba described in DE10211617
a a DNA fragment encoding an oleosin such as oleosin 3 (SEQ ID No 10 from amino acid 1 to amino acid 143) in, fused in the reading frame with
a DNA region encoding WS from mouse (SEQ ID 12) or a DNA region encoding WS from mouse tagged with YFP (SEQ ID No 14)
a 3′ transcription termination and polyadenylation region from the 3′ end of the nopaline synthase gene (3′nos: sequence including the 3′ untranslated region of the nopaline synthase gene from the T-DNA of pTiT37 of Agrobacterium tumefaciens (Depicker et al., 1982, Journal of Molecular and Applied Genetics, 1, 561-573)

These chimeric genes are introduced in a T-DNA vector, between the left and right border sequences from the T-DNA, together with a selectable marker gene such as a marker providing resistance to the herbicide phosphinotricin or a marker providing tolerance to glyphosate or a antibiotic resistance marker such as plant expressible nptII or hygromycin phosphotransferase.

The T-DNA vectors may also comprise both a McFar1 derived oleosin fusion encoding chimeric gene and a MmWS-oleosin encoding chimeric gene.

The T-DNA vectors are introduced in Agrobacterium comprising helper Ti-plasmid as standard in the art and used to generate transformed Arabidopsis and Brassica napus plants.

Plants are transformed, crossed or co-transformed to generate transgenic plants comprising MmFar1-oleosin and MmWS - oleo sin or MmFar1c-oleosin and MmWS-oleosin. Lipids are extracted from seeds and run on TLC. Short chain wax ester production is observed.

### REFERENCES

Abell, B. M., Holbrook, L. A., Abensen, M., Murphy, D. J., Hills, M. J., Moloney, M. M., 1997. Role of the proline knot motif in oleosin endoplasmatic reticulum topology and oil body targeting. Plant Cell 8, 1481-1493.

Brandizzi, F., Snapp, E. L., Roberts, A. G., Lippincott-Schwartz, J., Hawes, C., 2002. Membrane protein transport between the endoplasmic reticulum and the Golgi in tobacco leaves is energy dependent but cytoskeleton independent: evidence from selective photobleaching. Plant Cell 14, 1293-1309.

Cheng, J. B., Russell, D. W., 2004a. Mammalian wax biosynthesis. I. Identification of two fatty acyl-Coenzyme A reductases with different substrate specificities and tissue distributions. Journal of Biological Chemistry 279, 37789-37797.

Cheng, J. B., Russell, D. W., 2004b. Mammalian Wax Biosynthesis. II. Expression Cloning of Wax synthase cDNAs encoding a member of the acyltransferase enzyme family. Journal of Biological Chemistry 279, 37798-37807.

Dyer, J. M., Stymne, S., Green, A. G., Carlsson, A. S., 2008. High-value oils from plants. The Plant journal: cell and molecular biology 54, 640-655.

Fang, Y., Morrell, J. C., Jones, J. M., Gould, S. J., 2004. PEX3 functions as a PEX19 docking factor in the import of class I peroxisomal membrane proteins. The Journal of Cell Biology 164.

Fitzgerald, M., Murphy, R. C., 2007. Electrospray mass spectrometry of human hair wax esters. Journal of Lipid Research 48, 1231-1246.

Fransen, M., Wylin, T., Brees, C., Mannaerts, G. P., Van Veldhoven, P. P., 2001. Human Pex19p Binds Peroxisomal Integral Membrane Proteins at Regions Distinct from Their Sorting Sequences. . Molecular and Cellular Biology 21, 4413-4424.

Fukuda, M., Viitala, J., Matteson, J., Carlsson, S. R., 1988. Cloning of cDNAs encoding human lysosomal membrane glycoproteins, h-lamp-1 and h-lamp-2. Comparison of their deduced amino acid sequences. Journal of Biological Chemistry 263, 18920-18928.

Fulda, M., Shockey, J., Werber, M., Wolter, F. P., Heinz, E., 2002. Two long-chain acyl-CoA synthetases from Arabidopsis thaliana involved in peroxisomal fatty acid B-oxidation. The Plant Journal 32, 93-103.

Hadden, D. A., Phillipson, B. A., Johnston, K. A., Brown, L.-A., Manfield, I. W., El-Shami, M., Sparkes, I. A., Baker, A., 2006. Arabidopsis PEX19 is a dimeric protein that binds to peroxin PEX10. Molecular Membrane Biology 23, 325-336.

Heiland, I., Erdmann, R., 2005. Biogenesis of peroxisomes. Topogenesis of the peroxisomal membrane and matrix proteins. FEBS Journal 272, 2362-2372.

Hoffmann, M., Hornung, E., Busch, S., Kassner, N., Ternes, P., Braus, G. H., Feussner, I., 2007. A Small membrane-peripheral region Close to the Active Center Determines Regioselectivity of Membrane-bound Fatty Acid Desaturases from Aspergillus nidulans. The Journal of Biological Chemistry 282, 26666-26674.

Huang, A. H. C., 1992. Oil Bodies and Oleosins in Seeds. Annual Review Plant Physiology Plant Molecular Biology 43, 177-200. Inoue, H., Niojima, H., Okayama, H., 1990. High efficiency transformation of Escherichia coli with plasmids. Gene 96, 23-28.

Iyengar, B. T., Schlenk, H., 1969. Melting Points of Synthetic Wax Esters. Lipids 4, 28-30.

Jetter, R., Kunst, L., 2008. Plant surface lipid biosynthetic pathways and their utility for metabolic engineering of waxes and hydroarbon biofuels. Plant Journal 54, 670- 683.

Kunst, L., Samuels, A. L., 2003. Biosynthesis and secretion of plant cuticular wax. Progress of Lipid Research 42, 51-80.

Lardizabal, K., Metz, J. G., Sakamoto, T., Hutton, W. C., Pollard, M. R., Lassner, M. W., 2000. Purification of a Jojoba Embryo Wax Synthase, Cloning of its cDNA, and Production of High Levels of Wax in Seeds of Transgenic Arabidopsis. Plant Physiology 122, 645-655.

Lassner, M. W., Lardizabal, K., Metz, J. G., 1996. A jojoba b-ketoacyl-CoA synthase cDNA complements the canola fatty acid elongation mutation in transgenic plants. Plant Cell 8, 281-292.

Lassner, M. W., Lardizabal, K., Metz, J. G. (Eds.), 1999. Producing Wax Esters in Transgenic Plants by Expression of Genes Derived from Jojoba. ASHS Press, Alexandria, Va.

Matsuzono, Y., Fujiki, Y., 2006. In Vitro Transport of Membrane Proteins to Peroxisomes by Shuttling Receptor Pexl9p. Journal of Biological Chemistry 28, 36-42.

Millar, A. A., Kunst, L., 1997. Very-long-chain fatty acid biosynthesis is controlled through the expression and specificity of the condensing enzyme. Plant Journal 12, 121-131.

Pollard, M. R., McKeon, T., Gupta, L. M., Stumpf, P. K., 1979. Studies on biosynthesis of waxes by developing Jojoba seed. 2. The demonstration of wax biosynthesis by cell-free homogenates. Lipids 14, 651-662.

Sanger, F., Nicklen, S., Coulson, A. R., 1977. DNA sequencing with chain-terminating inhibitors. Biotechnology 24, 104-108

Stryer, L. (Ed.), 1995. Biochemie. Spektrum Akademischer Verlag Heidelberg, Heidelberg.

Todd, J., Post-Beittenmiller, D., 1999. KCS1 encodes a fatty acid elongase 3-ketoacyl-CoA synthase affecting wax biosynthesis in Arabidopsis thaliana. Plant Journal 17, 119-130.

von Wettstein-Knowles, P. (Ed.), 1982. Biosynthesis of epicuticular lipids as analysed with the aid of gene mutations in barley. Elsevier, Amsterdam.

Vrkoslav, V., Mikova, R., 2009. Characterization of natural wax esters by MALDI-TOF mass spectrometry. Journal of mass spectrometry 44, 101-110.

Xu, X., Dietrich, C. R., Lessire, R., Nikolau, B. J., Schnable, P. S., 2002. The endoplasmic reticulum-associated maize GL8 protein is a component of the acylcoenzyme A elongase involved in the production of cuticular waxes. Plant Physiology 128, 924-934.

Zheng, H., Rowland, 0., Kunst, L., 2005. Disruptions of the Arabidopsis enoyl-CoA reductase gene reveal an essential role for very-long-chain fatty acid synthesis in cell expansion during plant morphogenesis. Plant Cell 17.

Acknowledgment

The work leading to this invention has received funding from the European Community's Seventh Framework Programme FP7/2007-13 under agreement number 211400.

1. A method for modulating lipid biosynthesis in a eukaryotic organism comprising the step of providing to cells of said organism at least ene two chimeric proteins, wherein each of said chimeric proteins comprises
a. a heterologous polypeptide targeting said proteins to a similar subdomain of an organelle in said cell operably linked to
b. a polypeptide involved in fatty acid metabolism or lipid metabolism;
wherein said chimeric proteins comprise a different polypeptide involved in fatty acid metabolism or lipid metabolism.

2. (canceled)

3. The method according to claim 1 wherein said heterologous polypeptide comprises a polypeptide sequence selected from an oleosin, an endoplasmatic retrieval signal from fatty acid desaturases, the N-terminal domain of LBLOX or the N-terminal domain of PLA.

4. The method according to claim 3, wherein said heterologous polypeptide comprises a polypeptide sequence having at least 80% homology to the amino acid sequence of SEQ ID No 10 from amino acid 1 to amino acid 143.

5. (canceled)

6. The method according to claim 1, wherein said at least two chimeric proteins comprise the same heterologous polypeptide.

7. (canceled)

8. The method according to claim 1, wherein said multitude of chimeric proteins comprises at least one chimeric protein comprising a fatty acyl-CoA reductase and at least another chimeric protein comprising a wax ester synthase.

9. (canceled)

10. The method according to claim 8, wherein said fatty acyl-CoA reductase is derived from mouse and preferably has an amino acid sequence having about 80% sequence identity to the amino acid sequence of SEQ ID No 6, and wherein said wax ester synthase is derived from mouse and preferably has an amino acid sequence having about 80% sequence identity to the amino acid sequence of SEQ ID No 12.

11-12. (canceled)

13. The method according to claim 10, wherein said heterologous polypeptide targeting polypeptide comprises a polypeptide sequence having at least 80% homology to the amino acid sequence of SEQ ID No 10 from amino acid 1 to amino acid 143.

14. The method of claim 1 wherein said chimeric proteins are expressed from one or more DNA constructs comprising the following operably linked DNA fragments:
a. A promoter functional in cells of said eukaryotic organism
b. A DNA region encoding said chimeric protein
c. A transcription termination and/or polyadenylation region.

15. The method according to claim 1 wherein said eukaryotic organism is an oil producing plant.

16-29. (canceled)

30. A non-human eukaryotic organism comprising at least a first and a second DNA construct,
a. said first DNA construct comprising:
i. a first promoter functional in cells of said eukaryotic organism
ii. a first DNA region encoding a first chimeric protein comprising
1. A DNA region encoding a first heterologous polypeptide targeting said proteins to a particular subdomain of an organelle in said cell; operably linked to
2. A DNA region encoding a first polypeptide involved in fatty acid metabolism or lipid metabolism;
iii. a first transcription termination and/or polyadenylation region; and
b. said second DNA construct comprising
iv. a second promoter functional in cells of said eukaryotic organism
v. a second DNA region encoding a second chimeric protein comprising
3. A DNA region encoding a second heterologous polypeptide targeting said proteins to said particular subdomain of an organelle in said cell; operably linked to
4. A DNA region encoding a second polypeptide involved in fatty acid metabolism or lipid metabolism;
vi. a first transcription termination and/or polyadenylation region;
c. wherein said first and second heterologous polypeptide may be the same or different and wherein said first and second polypeptide involved in fatty acid metabolism or lipid metabolism are different polypeptides.

31. (canceled)

32. The organism according to claim 30, wherein said first and said second heterologous polypeptide comprise a polypeptide sequence selected from an oleosin, an endoplasmatic retrieval signal from fatty acid desaturases, the N-terminal domain of LBLOX or the N-terminal domain of PLA.

33. The organism according to claim 32, wherein said first and second heterologous polypeptide comprises a polypeptide sequence having at least 80% homology to the amino acid sequence of SEQ ID No 10 from amino acid 1 to amino acid 143.

34. The organism according to claim 30, wherein said first polypeptide involved in fatty acid or lipid metabolism comprises the amino acid sequence of a fatty acyl-CoA reductase and wherein said second polypeptide involved in fatty acid or lipid metabolism comprises the amino acid sequence of a wax ester synthase.

35. (canceled)

36. The organism according to claim 34, wherein said fatty acyl-CoA reductase is derived from mouse and preferably has an amino acid sequence having about 80% sequence identity to the amino acid sequence of SEQ ID No 6, and wherein said wax ester synthase is derived from mouse and preferably has an amino acid sequence having about 80% sequence identity to the amino acid sequence of SEQ ID No 12.

37. (canceled)

38. The organism according to claim 30 wherein said eukaryotic organism is an oil producing plant.

39-40. (canceled)


SEQUENCE LISTING (16 sequences)

SEQ ID NO: 1 - DNA, 1548 bp, Artificial
  mouse fatty acyl CoA reductase
atggtttccatcccagagtactatgagggaaagaacatcttgctaactggtgcaactggatttcttgggaaggttttgcttgagaagctgctaagatcttgccctagagtcaactccgtttacgttctagtcagacaaaaggctggacaaacacctcaagaaagagttgaggagatcttgtcaagcaagctctttgatagactgcgtgatgagaatccagatttccgagagaagatcatcgccatcaactctgaattgacccaacctaagttagcgctttctgaagaggataaggagatcatcatcgactcaacgaacgttatcttccattgcgctgcaacagttcgtttcaatgagaacctacgggatgctgttcaattgaacgtcattgctacgagacaactgatcttactcgctcagcagatgaagaacctcgaagtcttcatgcacgttagtactgcttacgcatactgtaaccgcaagcacatcgatgaagttgtttacccacctccagttgatcctaagaagttgatcgactctcttgagtggatggatgatggacttgtgaacgacataacaccaaagcttatcggagacagaccaaacacttacatctacactaaggcactggctgagtatgttgttcaacaagagggagctaagttgaacgttgcaatcgttagaccgtctattgttggagcttcatggaaagaaccattcccaggatggattgacaacttcaatggtccatctggacttttcattgcagctggcaagggtatcctcagaactatgagagcaagtaacaacgcacttgcagatcttgttcctgttgacgttgtcgttaacacctcattagcagctgcatggtattctggagttaacagacccaggaacatcatggtgtacaactgtacaacgggatctacaaacccttttcattggggtgaagttgagtatcacgtcatctctaccttcaagagaaaccctcttgagcaagctttcagaagacctaacgtgaaccttacctccaatcatctactctaccactactggattgctgtttctcataaggctcctgctttcttgtacgacatctaccttcgaatgactggtagaagtcctcggatgatgaagaccattacacgactacacaaggctatggtcttccttgagtacttcacctcaaactcatgggtttggaacactgacaacgttaacatgctcatgaaccagcttaaccctgaggacaagaagacgttcaacattgatgttcgacagcttcactgggctgagtacatagagaactactgtatggggacgaagaagtacgttcttaacgaagagatgtctggacttccagcagctagaaaacatctgaacaagcttcgcaacatcagatacggtttcaacaccatcctggttatccttatctggaggatcttcatcgctagaagtcaaatggcgagaaacatctggtacttcgttgtttcgctgtgctacaagttcctttcttacttcagagcctcttccacaatgcgatactaa

SEQ ID NO: 2 - protein, 515 aa, Artificial
  Synthetic Construct
MVSIPEYYEGKNILLTGATGFLGKVLLEKLLRSCPRVNSVYVLVRQKAGQTPQERVEEILSSKLFDRLRDENPDFREKIIAINSELTQPKLALSEEDKEIIIDSTNVIFHCAATVRFNENLRDAVQLNVIATRQLILLAQQMKNLEVFMHVSTAYAYCNRKHIDEVVYPPPVDPKKLIDSLEWMDDGLVNDITPKLIGDRPNTYIYTKALAEYVVQQEGAKLNVAIVRPSIVGASWKEPFPGWIDNFNGPSGLFIAAGKGILRTMRASNNALADLVPVDVVVNTSLAAAWYSGVNRPRNIMVYNCTTGSTNPFHWGEVEYHVISTFKRNPLEQAFRRPNVNLTSNHLLYHYWIAVSHKAPAFLYDIYLRMTGRSPRMMKTITRLHKAMVFLEYFTSNSWVWNTDNVNMLMNQLNPEDKKTFNIDVRQLHWAEYIENYCMGTKKYVLNEEMSGLPAARKHLNKLRNIRYGFNTILVILIWRIFIARSQMARNIWYFVVSLCYKFLSYFRASSTMRY

SEQ ID NO: 3 - DNA, 2274 bp, Artificial
  mouse fatty acyl CoA reductase tagged with mCherry
ggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcatggtttccatcccagagtactatgagggaaagaacatcttgctaactggtgcaactggatttcttgggaaggttttgcttgagaagctgctaagatcttgccctagagtcaactccgtttacgttctagtcagacaaaaggctggacaaacacctcaagaaagagttgaggagatcttgtcaagcaagctctttgatagactgcgtgatgagaatccagatttccgagagaagatcatcgccatcaactctgaattgacccaacctaagttagcgctttctgaagaggataaggagatcatcatcgactcaacgaacgttatcttccattgcgctgcaacagttcgtttcaatgagaacctacgggatgctgttcaattgaacgtcattgctacgagacaactgatcttactcgctcagcagatgaagaacctcgaagtcttcatgcacgttagtactgcttacgcatactgtaaccgcaagcacatcgatgaagttgtttacccacctccagttgatcctaagaagttgatcgactctcttgagtggatggatgatggacttgtgaacgacataacaccaaagcttatcggagacagaccaaacacttacatctacactaaggcactggctgagtatgttgttcaacaagagggagctaagttgaacgttgcaatcgttagaccgtctattgttggagcttcatggaaagaaccattcccaggatggattgacaacttcaatggtccatctggacttttcattgcagctggcaagggtatcctcagaactatgagagcaagtaacaacgcacttgcagatcttgttcctgttgacgttgtcgttaacacctcattagcagctgcatggtattctggagttaacagacccaggaacatcatggtgtacaactgtacaacgggatctacaaacccttttcattggggtgaagttgagtatcacgtcatctctaccttcaagagaaaccctcttgagcaagctttcagaagacctaacgtgaaccttacctccaatcatctactctaccactactggattgctgtttctcataaggctcctgctttcttgtacgacatctaccttcgaatgactggtagaagtcctcggatgatgaagaccattacacgactacacaaggctatggtcttccttgagtacttcacctcaaactcatgggtttggaacactgacaacgttaacatgctcatgaaccagcttaaccctgaggacaagaagacgttcaacattgatgttcgacagcttcactgggctgagtacatagagaactactgtatggggacgaagaagtacgttcttaacgaagagatgtctggacttccagcagctagaaaacatctgaacaagcttcgcaacatcagatacggtttcaacaccatcctggttatccttatctggaggatcttcatcgctagaagtcaaatggcgagaaacatctggtacttcgttgtttcgctgtgctacaagttcctttcttacttcagagcctcttccacaatgcgatactaactcgag

SEQ ID NO: 4 - protein, 753 aa, Artificial
  Synthetic Construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFMVSIPEYYEGKNILLTGATGFLGKVLLEKLLRSCPRVNSVYVLVRQKAGQTPQERVEEILSSKLFDRLRDENPDFREKIIAINSELTQPKLALSEEDKEIIIDSTNVIFHCAATVRFNENLRDAVQLNVIATRQLILLAQQMKNLEVFMHVSTAYAYCNRKHIDEVVYPPPVDPKKLIDSLEWMDDGLVNDITPKLIGDRPNTYIYTKALAEYVVQQEGAKLNVAIVRPSIVGASWKEPFPGWIDNFNGPSGLFIAAGKGILRTMRASNNALADLVPVDVVVNTSLAAAWYSGVNRPRNIMVYNCTTGSTNPFHWGEVEYHVISTFKRNPLEQAFRRPNVNLTSNHLLYHYWIAVSHKAPAFLYDIYLRMTGRSPRMMKTITRLHKAMVFLEYFTSNSWVWNTDNVNMLMNQLNPEDKKTFNIDVRQLHWAEYIENYCMGTKKYVLNEEMSGLPAARKHLNKLRNIRYGFNTILVILIWRIFIARSQMARNIWYFVVSLCYKFLSYFRASSTMRY

SEQ ID NO: 5 - DNA, 1404 bp, Artificial
  mouse fatty acyl CoA reductase truncated
atggtttccatcccagagtactatgagggaaagaacatcttgctaactggtgcaactggatttcttgggaaggttttgcttgagaagctgctaagatcttgccctagagtcaactccgtttacgttctagtcagacaaaaggctggacaaacacctcaagaaagagttgaggagatcttgtcaagcaagctctttgatagactgcgtgatgagaatccagatttccgagagaagatcatcgccatcaactctgaattgacccaacctaagttagcgctttctgaagaggataaggagatcatcatcgactcaacgaacgttatcttccattgcgctgcaacagttcgtttcaatgagaacctacgggatgctgttcaattgaacgtcattgctacgagacaactgatcttactcgctcagcagatgaagaacctcgaagtcttcatgcacgttagtactgcttacgcatactgtaaccgcaagcacatcgatgaagttgtttacccacctccagttgatcctaagaagttgatcgactctcttgagtggatggatgatggacttgtgaacgacataacaccaaagcttatcggagacagaccaaacacttacatctacactaaggcactggctgagtatgttgttcaacaagagggagctaagttgaacgttgcaatcgttagaccgtctattgttggagcttcatggaaagaaccattcccaggatggattgacaacttcaatggtccatctggacttttcattgcagctggcaagggtatcctcagaactatgagagcaagtaacaacgcacttgcagatcttgttcctgttgacgttgtcgttaacacctcattagcagctgcatggtattctggagttaacagacccaggaacatcatggtgtacaactgtacaacgggatctacaaacccttttcattggggtgaagttgagtatcacgtcatctctaccttcaagagaaaccctcttgagcaagctttcagaagacctaacgtgaaccttacctccaatcatctactctaccactactggattgctgtttctcataaggctcctgctttcttgtacgacatctaccttcgaatgactggtagaagtcctcggatgatgaagaccattacacgactacacaaggctatggtcttccttgagtacttcacctcaaactcatgggtttggaacactgacaacgttaacatgctcatgaaccagcttaaccctgaggacaagaagacgttcaacattgatgttcgacagcttcactgggctgagtacatagagaactactgtatggggacgaagaagtacgttcttaacgaagagatgtctggacttccagcagctagaaaacatctgaacaagcttcgcaacatcagataa

SEQ ID NO: 6 - protein, 467 aa, Artificial
  Synthetic Construct
MVSIPEYYEGKNILLTGATGFLGKVLLEKLLRSCPRVNSVYVLVRQKAGQTPQERVEEILSSKLFDRLRDENPDFREKIIAINSELTQPKLALSEEDKEIIIDSTNVIFHCAATVRFNENLRDAVQLNVIATRQLILLAQQMKNLEVFMHVSTAYAYCNRKHIDEVVYPPPVDPKKLIDSLEWMDDGLVNDITPKLIGDRPNTYIYTKALAEYVVQQEGAKLNVAIVRPSIVGASWKEPFPGWIDNFNGPSGLFIAAGKGILRTMRASNNALADLVPVDVVVNTSLAAAWYSGVNRPRNIMVYNCTTGSTNPFHWGEVEYHVISTFKRNPLEQAFRRPNVNLTSNHLLYHYWIAVSHKAPAFLYDIYLRMTGRSPRMMKTITRLHKAMVFLEYFTSNSWVWNTDNVNMLMNQLNPEDKKTFNIDVRQLHWAEYIENYCMGTKKYVLNEEMSGLPAARKHLNKLRNIR

SEQ ID NO: 7 - DNA, 2130 bp, Artificial
  mouse fatty acyl CoA reductase tagged with mCherry
ggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcatggtttccatcccagagtactatgagggaaagaacatcttgctaactggtgcaactggatttcttgggaaggttttgcttgagaagctgctaagatcttgccctagagtcaactccgtttacgttctagtcagacaaaaggctggacaaacacctcaagaaagagttgaggagatcttgtcaagcaagctctttgatagactgcgtgatgagaatccagatttccgagagaagatcatcgccatcaactctgaattgacccaacctaagttagcgctttctgaagaggataaggagatcatcatcgactcaacgaacgttatcttccattgcgctgcaacagttcgtttcaatgagaacctacgggatgctgttcaattgaacgtcattgctacgagacaactgatcttactcgctcagcagatgaagaacctcgaagtcttcatgcacgttagtactgcttacgcatactgtaaccgcaagcacatcgatgaagttgtttacccacctccagttgatcctaagaagttgatcgactctcttgagtggatggatgatggacttgtgaacgacataacaccaaagcttatcggagacagaccaaacacttacatctacactaaggcactggctgagtatgttgttcaacaagagggagctaagttgaacgttgcaatcgttagaccgtctattgttggagcttcatggaaagaaccattcccaggatggattgacaacttcaatggtccatctggacttttcattgcagctggcaagggtatcctcagaactatgagagcaagtaacaacgcacttgcagatcttgttcctgttgacgttgtcgttaacacctcattagcagctgcatggtattctggagttaacagacccaggaacatcatggtgtacaactgtacaacgggatctacaaacccttttcattggggtgaagttgagtatcacgtcatctctaccttcaagagaaaccctcttgagcaagctttcagaagacctaacgtgaaccttacctccaatcatctactctaccactactggattgctgtttctcataaggctcctgctttcttgtacgacatctaccttcgaatgactggtagaagtcctcggatgatgaagaccattacacgactacacaaggctatggtcttccttgagtacttcacctcaaactcatgggtttggaacactgacaacgttaacatgctcatgaaccagcttaaccctgaggacaagaagacgttcaacattgatgttcgacagcttcactgggctgagtacatagagaactactgtatggggacgaagaagtacgttcttaacgaagagatgtctggacttccagcagctagaaaacatctgaacaagcttcgcaacatcagataactcgag

SEQ ID NO: 8 - protein, 705 aa, Artificial
  Synthetic Construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFMVSIPEYYEGKNILLTGATGFLGKVLLEKLLRSCPRVNSVYVLVRQKAGQTPQERVEEILSSKLFDRLRDENPDFREKIIAINSELTQPKLALSEEDKEIIIDSTNVIFHCAATVRFNENLRDAVQLNVIATRQLILLAQQMKNLEVFMHVSTAYAYCNRKHIDEVVYPPPVDPKKLIDSLEWMDDGLVNDITPKLIGDRPNTYIYTKALAEYVVQQEGAKLNVAIVRPSIVGASWKEPFPGWIDNFNGPSGLFIAAGKGILRTMRASNNALADLVPVDVVVNTSLAAAWYSGVNRPRNIMVYNCTTGSTNPFHWGEVEYHVISTFKRNPLEQAFRRPNVNLTSNHLLYHYWIAVSHKAPAFLYDIYLRMTGRSPRMMKTITRLHKAMVFLEYFTSNSWVWNTDNVNMLMNQLNPEDKKTFNIDVRQLHWAEYIENYCMGTKKYVLNEEMSGLPAARKHLNKLRNIR

SEQ ID NO: 9 - DNA, 2559 bp, Artificial
  mouse fatty acyl CoA reductase truncated, tagged with mCherry and fused to oleosin 3At
gtcgacatggcggaccaaacaagaacccatcacgagatgataagccgagacagtacccaagaggcccatccgaaggccaggcagatggtgaaggcagcaaccgctgtcacagccggtggatccctacttgtcctctccggcttaacactcgctggaacagtcatcgcactcacggtggctactcctctcctcgtcatcttcagccccgtcttggttccagcagtggtaaccgttgctctcatcattaccggattccttgcatccggtggctttggaatagccgccattaccgccttctcttggctctacaggcacatgacgggatctggatcggataagatagagaatgctcggatgaaggttggaagcagagtgcaggatactaagtatgggcagcacaacattggagtccaacaccaacaagtttctggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcatggtttccatcccagagtactatgagggaaagaacatcttgctaactggtgcaactggatttcttgggaaggttttgcttgagaagctgctaagatcttgccctagagtcaactccgtttacgttctagtcagacaaaaggctggacaaacacctcaagaaagagttgaggagatcttgtcaagcaagctctttgatagactgcgtgatgagaatccagatttccgagagaagatcatcgccatcaactctgaattgacccaacctaagttagcgctttctgaagaggataaggagatcatcatcgactcaacgaacgttatcttccattgcgctgcaacagttcgtttcaatgagaacctacgggatgctgttcaattgaacgtcattgctacgagacaactgatcttactcgctcagcagatgaagaacctcgaagtcttcatgcacgttagtactgcttacgcatactgtaaccgcaagcacatcgatgaagttgtttacccacctccagttgatcctaagaagttgatcgactctcttgagtggatggatgatggacttgtgaacgacataacaccaaagcttatcggagacagaccaaacacttacatctacactaaggcactggctgagtatgttgttcaacaagagggagctaagttgaacgttgcaatcgttagaccgtctattgttggagcttcatggaaagaaccattcccaggatggattgacaacttcaatggtccatctggacttttcattgcagctggcaagggtatcctcagaactatgagagcaagtaacaacgcacttgcagatcttgttcctgttgacgttgtcgttaacacctcattagcagctgcatggtattctggagttaacagacccaggaacatcatggtgtacaactgtacaacgggatctacaaacccttttcattggggtgaagttgagtatcacgtcatctctaccttcaagagaaaccctcttgagcaagctttcagaagacctaacgtgaaccttacctccaatcatctactctaccactactggattgctgtttctcataaggctcctgctttcttgtacgacatctaccttcgaatgactggtagaagtcctcggatgatgaagaccattacacgactacacaaggctatggtcttccttgagtacttcacctcaaactcatgggtttggaacactgacaacgttaacatgctcatgaaccagcttaaccctgaggacaagaagacgttcaacattgatgttcgacagcttcactgggctgagtacatagagaactactgtatggggacgaagaagtacgttcttaacgaagagatgtctggacttccagcagctagaaaacatctgaacaagcttcgcaacatcagataactcgag

SEQ ID NO: 10 - protein, 848 aa, Artificial
  Synthetic Construct
MADQTRTHHEMISRDSTQEAHPKARQMVKAATAVTAGGSLLVLSGLTLAGTVIALTVATPLLVIFSPVLVPAVVTVALIITGFLASGGFGIAAITAFSWLYRHMTGSGSDKIENARMKVGSRVQDTKYGQHNIGVQHQQVSGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFMVSIPEYYEGKNILLTGATGFLGKVLLEKLLRSCPRVNSVYVLVRQKAGQTPQERVEEILSSKLFDRLRDENPDFREKIIAINSELTQPKLALSEEDKEIIIDSTNVIFHCAATVRFNENLRDAVQLNVIATRQLILLAQQMKNLEVFMHVSTAYAYCNRKHIDEVVYPPPVDPKKLIDSLEWMDDGLVNDITPKLIGDRPNTYIYTKALAEYVVQQEGAKLNVAIVRPSIVGASWKEPFPGWIDNFNGPSGLFIAAGKGILRTMRASNNALADLVPVDVVVNTSLAAAWYSGVNRPRNIMVYNCTTGSTNPFHWGEVEYHVISTFKRNPLEQAFRRPNVNLTSNHLLYHYWIAVSHKAPAFLYDIYLRMTGRSPRMMKTITRLHKAMVFLEYFTSNSWVWNTDNVNMLMNQLNPEDKKTFNIDVRQLHWAEYIENYCMGTKKYVLNEEMSGLPAARKHLNKLRNIR

SEQ ID NO: 11 - DNA, 1002 bp, Artificial
  Wax ester synthase from mouse
atgttctggcctactaagaaggaccttaagactgcaatggaggttttcgctcttttccaatgggctttgtctgcactcgttatcgttactaccgtcatcatcgtgaacctgtaccttgttgtgttcacttcatactggccagttaccgttttgatgctaacatggctggcattcgattggaaaacacctgaaagaggaggtagaaggttcacatgtgtgagaaagtggcgtctttggaaacactactctgactacttccctcttaagatggtgaagacgaaggatatctcccctgatagaaactacatacttgtctgtcacccacatggtcttatggcacatagttgtttcggacatttcgctaccgatactacaggattcagtaagaccttccctggtatcacaccttacatgcttactcttggagctttcttctgggttcctttcttgagagattacgtcatgtcaaccggaagttgttctgtgtccagaagctctatggacttcttgcttacgcaaaagggtacaggaaacatgcttgttgtcgttgttggtggacttgctgaatgtagatactctacaccaggctcaacaacccttttcctaaagaagaggcagggatttgttcgtacagcactaaagcatggggtttctttgatccctgcttatgcatttggagagactgacctctacgatcaacatatcttcactcctggaggattcgtcaacagattccaaaagtggttccagaagatggttcacatctacccatgtgctttctatggaagaggtctgaccaagaattcatggggtcttttgccttactcacaaccagttactacagttgttggagagccattaccgttgcctaagatagaaaaccctagcgaagagattgttgccaagtatcacaccctttacatcgatgctctcagaaagctattcgaccagcacaagactaagttcggaatctctgagacacaagagctggttatcgtctaa

SEQ ID NO: 12 - protein, 333 aa, Artificial
  Synthetic Construct
MFWPTKKDLKTAMEVFALFQWALSALVIVTTVIIVNLYLVVFTSYWPVTVLMLTWLAFDWKTPERGGRRFTCVRKWRLWKHYSDYFPLKMVKTKDISPDRNYILVCHPHGLMAHSCFGHFATDTTGFSKTFPGITPYMLTLGAFFWVPFLRDYVMSTGSCSVSRSSMDFLLTQKGTGNMLVVVVGGLAECRYSTPGSTTLFLKKRQGFVRTALKHGVSLIPAYAFGETDLYDQHIFTPGGFVNRFQKWFQKMVHIYPCAFYGRGLTKNSWGLLPYSQPVTTVVGEPLPLPKIENPSEEIVAKYHTLYIDALRKLFDQHKTKFGISETQELVIV

SEQ ID NO: 13 - DNA, 1731 bp, Artificial
  Wax ester synthase from mouse tagged with YFP
actagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagatgttctggcctactaagaaggaccttaagactgcaatggaggttttcgctcttttccaatgggctttgtctgcactcgttatcgttactaccgtcatcatcgtgaacctgtaccttgttgtgttcacttcatactggccagttaccgttttgatgctaacatggctggcattcgattggaaaacacctgaaagaggaggtagaaggttcacatgtgtgagaaagtggcgtctttggaaacactactctgactacttccctcttaagatggtgaagacgaaggatatctcccctgatagaaactacatacttgtctgtcacccacatggtcttatggcacatagttgtttcggacatttcgctaccgatactacaggattcagtaagaccttccctggtatcacaccttacatgcttactcttggagctttcttctgggttcctttcttgagagattacgtcatgtcaaccggaagttgttctgtgtccagaagctctatggacttcttgcttacgcaaaagggtacaggaaacatgcttgttgtcgttgttggtggacttgctgaatgtagatactctacaccaggctcaacaacccttttcctaaagaagaggcagggatttgttcgtacagcactaaagcatggggtttctttgatccctgcttatgcatttggagagactgacctctacgatcaacatatcttcactcctggaggattcgtcaacagattccaaaagtggttccagaagatggttcacatctacccatgtgctttctatggaagaggtctgaccaagaattcatggggtcttttgccttactcacaaccagttactacagttgttggagagccattaccgttgcctaagatagaaaaccctagcgaagagattgttgccaagtatcacaccctttacatcgatgctctcagaaagctattcgaccagcacaagactaagttcggaatctctgagacacaagagctggttatcgtctaagagctc

SEQ ID NO: 14 - protein, 572 aa, Artificial
  Synthetic Construct
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKMFWPTKKDLKTAMEVFALFQWALSALVIVTTVIIVNLYLVVFTSYWPVTVLMLTWLAFDWKTPERGGRRFTCVRKWRLWKHYSDYFPLKMVKTKDISPDRNYILVCHPHGLMAHSCFGHFATDTTGFSKTFPGITPYMLTLGAFFWVPFLRDYVMSTGSCSVSRSSMDFLLTQKGTGNMLVVVVGGLAECRYSTPGSTTLFLKKRQGFVRTALKHGVSLIPAYAFGETDLYDQHIFTPGGFVNRFQKWFQKMVHIYPCAFYGRGLTKNSWGLLPYSQPVTTVVGEPLPLPKIENPSEEIVAKYHTLYIDALRKLFDQHKTKFGISETQELVIV

SEQ ID NO: 15 - DNA, 2154 bp, Artificial
  Wax ester synthase from mouse tagged with YFP fused to oleosin 3 At
actagtatggcggaccaaacaagaacccatcacgagatgataagccgagacagtacccaagaggcccatccgaaggccaggcagatggtgaaggcagcaaccgctgtcacagccggtggatccctacttgtcctctccggcttaacactcgctggaacagtcatcgcactcacggtggctactcctctcctcgtcatcttcagccccgtcttggttccagcagtggtaaccgttgctctcatcattaccggattccttgcatccggtggctttggaatagccgccattaccgccttctcttggctctacaggcacatgacgggatctggatcggataagatagagaatgctcggatgaaggttggaagcagagtgcaggatactaagtatgggcagcacaacattggagtccaacaccaacaagtttctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagatgttctggcctactaagaaggaccttaagactgcaatggaggttttcgctcttttccaatgggctttgtctgcactcgttatcgttactaccgtcatcatcgtgaacctgtaccttgttgtgttcacttcatactggccagttaccgttttgatgctaacatggctggcattcgattggaaaacacctgaaagaggaggtagaaggttcacatgtgtgagaaagtggcgtctttggaaacactactctgactacttccctcttaagatggtgaagacgaaggatatctcccctgatagaaactacatacttgtctgtcacccacatggtcttatggcacatagttgtttcggacatttcgctaccgatactacaggattcagtaagaccttccctggtatcacaccttacatgcttactcttggagctttcttctgggttcctttcttgagagattacgtcatgtcaaccggaagttgttctgtgtccagaagctctatggacttcttgcttacgcaaaagggtacaggaaacatgcttgttgtcgttgttggtggacttgctgaatgtagatactctacaccaggctcaacaacccttttcctaaagaagaggcagggatttgttcgtacagcactaaagcatggggtttctttgatccctgcttatgcatttggagagactgacctctacgatcaacatatcttcactcctggaggattcgtcaacagattccaaaagtggttccagaagatggttcacatctacccatgtgctttctatggaagaggtctgaccaagaattcatggggtcttttgccttactcacaaccagttactacagttgttggagagccattaccgttgcctaagatagaaaaccctagcgaagagattgttgccaagtatcacaccctttacatcgatgctctcagaaagctattcgaccagcacaagactaagttcggaatctctgagacacaagagctggttatcgtctaagagctc

SEQ ID NO: 16 - protein, 713 aa, Artificial
  Synthetic Construct
MADQTRTHHEMISRDSTQEAHPKARQMVKAATAVTAGGSLLVLSGLTLAGTVIALTVATPLLVIFSPVLVPAVVTVALIITGFLASGGFGIAAITAFSWLYRHMTGSGSDKIENARMKVGSRVQDTKYGQHNIGVQHQQVSMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKMFWPTKKDLKTAMEVFALFQWALSALVIVTTVIIVNLYLVVFTSYWPVTVLMLTWLAFDWKTPERGGRRFTCVRKWRLWKHYSDYFPLKMVKTKDISPDRNYILVCHPHGLMAHSCFGHFATDTTGFSKTFPGITPYMLTLGAFFWVPFLRDYVMSTGSCSVSRSSMDFLLTQKGTGNMLVVVVGGLAECRYSTPGSTTLFLKKRQGFVRTALKHGVSLIPAYAFGETDLYDQHIFTPGGFVNRFQKWFQKMVHIYPCAFYGRGLTKNSWGLLPYSQPVTTVVGEPLPLPKIENPSEEIVAKYHTLYIDALRKLFDQHKTKFGISETQELVIV